(19) United States
(12) Patent Application Publication
(10) Pub. No.: US 2025/0116641 A1
(43) Pub. Date: Apr. 10, 2025

(54) Gas Detector

(71) Applicant: RIKEN KEIKI CO., LTD., Tokyo (JP)

(72) Inventors: Kazunori YAMADA, Kasukabe-shi (JP); Hideki KAWARAI, Kasukabe-shi (JP); Ryohei SHIRAISHI, Kasukabe-shi (JP); Muneyoshi KANEDA, Kasukabe-shi (JP)

(21) Appl. No.: 18/906,255

(22) Filed: Oct. 4, 2024

(30) Foreign Application Priority Data
Oct. 10, 2023 (JP) 2023-175124

(51) Int. Cl. G01N 33/00

(52) U.S. Cl. CPC G01N 33/0009

(57) ABSTRACT

A small gas detector capable of accommodating various gas detection techniques is provided. The gas detector includes a main body; a chamber that is separably disposed on the main body and includes a chamber-side inflow channel, a chamber-side outflow channel, and an internal space; a gas sensor that is separably disposed on the chamber; and a sensor base that is separably disposed on the gas sensor and the main body, and that includes a sensor receptacle connector detachably connected to the gas sensor and a main body receptacle connector detachably connected to the main body.

### BACKGROUND OF THE INVENTION

### Field of the Invention

The present invention relates to a gas detector for detecting gas.

### Description of Related Art

Some gas detectors are configured so that gas sensors (gas detection modules) for detecting gas can be replaced to detect a plurality of types of gases. Some gas detectors can simultaneously detect a plurality of types of gases by incorporating a plurality of types of gas sensors within a single gas detector device (see Patent Literature 1).

### BRIEF SUMMARY OF THE INVENTION

However, conventional gas detectors have had a problem that if a plurality of types of gas detection modules corresponding to various gas detection techniques are prepared in advance, all gas detection modules become large in size due to size standardization. As a result, the entire gas detectors become large in size.

In view of the foregoing circumstances, an object of the present invention is to provide a gas detector that can be reduced in size while accommodating various gas detection techniques.

The present invention related to the foregoing object is a gas detector for detecting gas, the gas detector including: a main body including a main body-side inflow channel through which the gas flows in and a main body-side outflow channel through which the gas flows out; a chamber that is separably disposed on the main body and includes a chamber-side inflow channel detachably connected to the main body-side inflow channel, a chamber-side outflow channel detachably connected to the main body-side outflow channel, and an internal space interposed between the chamber-side inflow channel and the chamber-side outflow channel; a gas sensor that is separably disposed on the chamber and includes a gas detection unit to be in contact with the gas passing through the internal space; and a sensor base that is separably disposed on the gas sensor and the main body, and that includes a sensor receptacle connector detachably connected to a sensor-side connector of the gas sensor and a main body receptacle connector detachably connected to a main body-side connector of the main body to thereby electrically connect the gas sensor and the main body.

Concerning the gas detector, the chamber may include a sensor insertion port into which the gas sensor is inserted. The gas sensor may include an annular sensor-side sealing surface capable of abutting against the sensor insertion port. The internal space may be enclosed by the sensor insertion port and the sensor-side sealing surface abutting against each other.

Concerning the gas detector, the chamber may include a chamber-side engagement portion. The sensor base may include a base-side engagement portion to be engaged with the chamber-side engagement portion. The chamber may be fixed to the sensor base by engaging the chamber-side engagement portion and the base-side engagement portion with each other. The chamber may be made movable relative to the sensor base by disengaging the chamber-side engagement portion and the base-side engagement portion.

Concerning the gas detector, the gas detector may include the gas sensor in multiple units. The gas detection units of the gas sensors may be in contact with the gas passing through the internal space of the chamber. The sensor base may include a plurality of sensor receptacle connectors corresponding to the gas sensors.

Concerning the gas detector, the sensor base may include a base-side computer. The base-side computer may include an output multiplexer processing unit that multiplexes outputs of the gas sensors and transmits the multiplexed outputs to the main body via the main body receptacle connector.

Concerning the gas detector, the gas sensors may each include a sensor-side computer. The sensor-side computer may include a normalization processing unit that normalizes the output of the gas detection unit.

Concerning the gas detector, the sensor base may include a base-side computer. The base-side computer may include an input demultiplexer processing unit that separates multiplexed input signals transmitted from the main body via the main body receptacle connector and distributes the separated input signals to the gas sensors.

According to the present invention, an entire gas detector can be reduced in size while accommodating various gas detection techniques.

### BRIEF DESCRIPTION OF THE DRAWINGS

These and other characteristics, features, and advantages of the present invention will become clear from the following description with reference to the accompanying drawings, wherein:

FIG. 1A is a front perspective view of a main body of a gas detector according to a first embodiment, as seen obliquely from above, and FIG. 1B is a rear perspective view of the main body of the gas detector, as seen obliquely from below;

FIG. 2A is a top view of the main body, FIG. 2B is a front view thereof, FIG. 2C is a right side view thereof, FIG. 2D is a left side view thereof, FIG. 2E is a bottom view thereof, and FIG. 2F is a rear view thereof;

FIG. 3 is a perspective view of the main body in a state in which a front cover is opened;

FIG. 4 is a front view of the main body with a front cover in an open state;

FIG. 5 is a left side view of the main body with a front cover and a left side cover omitted;

FIG. 6 is a perspective view of the main body with a pump disassembled;

FIG. 7 is a perspective view of the main body with a flow sensor disassembled;

FIG. 8 is a front view of the main body with the pump and the flow sensor dismounted;

FIG. 9A is a rear perspective view of a gas detection module of the gas detector according to the first embodiment, FIG. 9B is a left side view thereof, and FIG. 9C is a rear view thereof;

FIG. 10A is a rear perspective view of the gas detection module with a chamber opened as seen from below, FIG. 10B is a bottom view thereof, and FIG. 10C is a perspective view of the gas sensor disassembled;

FIG. 11A is a left side view of the gas detector with the main body and the gas detection module separated, and FIG. 11B is a left side view with the gas detection module mounted on the main body;

FIG. 12A is a block diagram illustrating an internal circuit configuration of the gas detector;

FIG. 12B is a block diagram illustrating a functional configuration of a main body-side computer, a base-side computer and a sensor-side computer of the gas detector;

FIG. 13A is a block diagram illustrating a modified example of the internal circuit configuration of the gas detector;

FIG. 13B is a block diagram illustrating a modified example of the functional configuration of the main body-side computer, the base-side computer and the sensor-side computer of the gas detector;

FIG. 14 is a diagram illustrating a determination table with which the main body-side computer of the gas detector determines the type of gas detection module;

FIG. 15 is a flow chart illustrating a procedures of a gas detection program of the gas detector;

FIG. 16A is a rear perspective view of a gas detection module of a gas detector according to a second embodiment, FIG. 16B is a left side view thereof, and FIG. 16C is a rear view thereof;

FIG. 17A is a rear perspective view of the gas detection module with a chamber opened as seen from below, and FIG. 17B is a perspective view of the gas sensor disassembled;

FIG. 18A is a left side view of the gas detector with a main body and the gas detection module separated, and FIG. 18B is a left side view with the gas detection module mounted on the main body;

FIG. 19 is a block diagram illustrating an internal circuit configuration of the gas detector;

FIG. 20 is a block diagram illustrating a functional configuration of a main body-side computer, a base-side computer, a first sensor-side computer, and a second sensor-side computer of the gas detector;

FIG. 21A is a rear perspective view of a chamber in a gas detection module of a gas detector according to a third embodiment as seen from above, FIG. 21B is a front view thereof, FIG. 21C is a right side view thereof, FIG. 21D is a left side view thereof, and FIG. 21E is a rear view thereof;

FIG. 22A is a front perspective view of a sensor unit in the gas detection module as seen obliquely from below, FIG. 22B is a rear perspective view thereof as seen obliquely from above, FIG. 22C is a rear view thereof, and FIG. 22D is a left side view thereof;

FIG. 23 is a rear view illustrating the gas detection module with the sensor unit and a chamber connected to each other;

FIG. 24A is a left side view illustrating the gas detector with a main body, the sensor unit, and the chamber separated, FIG. 24B is a left side view illustrating the main body with the chamber mounted, and FIG. 24C is a left side view illustrating the main body with the sensor unit mounted;

FIG. 25 is a block diagram illustrating an internal circuit configuration of the gas detector;

FIG. 26 is a block diagram illustrating a functional configuration of a main body-side computer and a unit-side computer of the gas detector; and

FIG. 27 is a diagram illustrating a determination table with which the base-side computer of the gas detector according to the second embodiment determines the type of gas sensor.

### DETAILED DESCRIPTION OF THE INVENTION

A description will now be made below with reference to the accompanying drawings in accordance with embodiments.

### First Embodiment

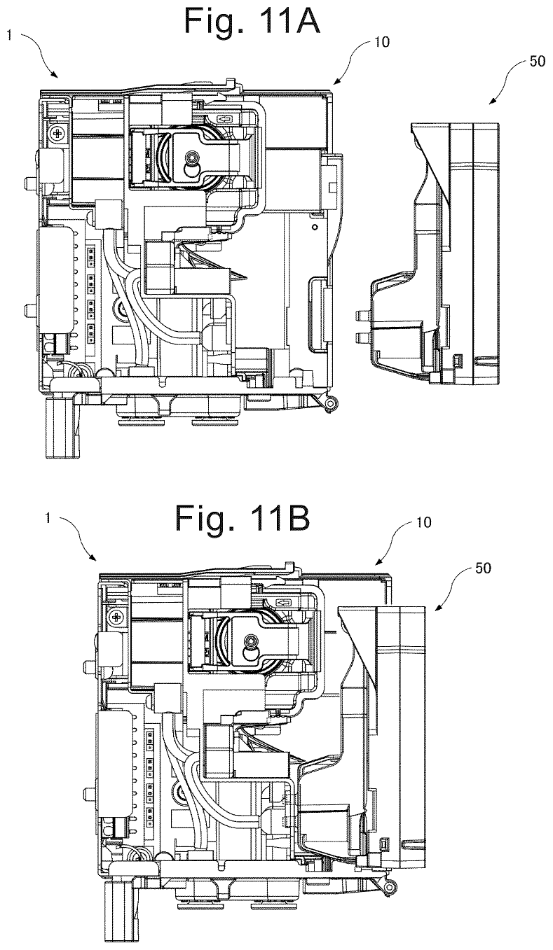

As shown in FIGS. 11A and 11B, a gas detector 1 according to a first embodiment includes a main body 10 and a gas detection module 50. The main body 10 and the gas detection module 50 are detachably connected.

### (Main Body Housing)

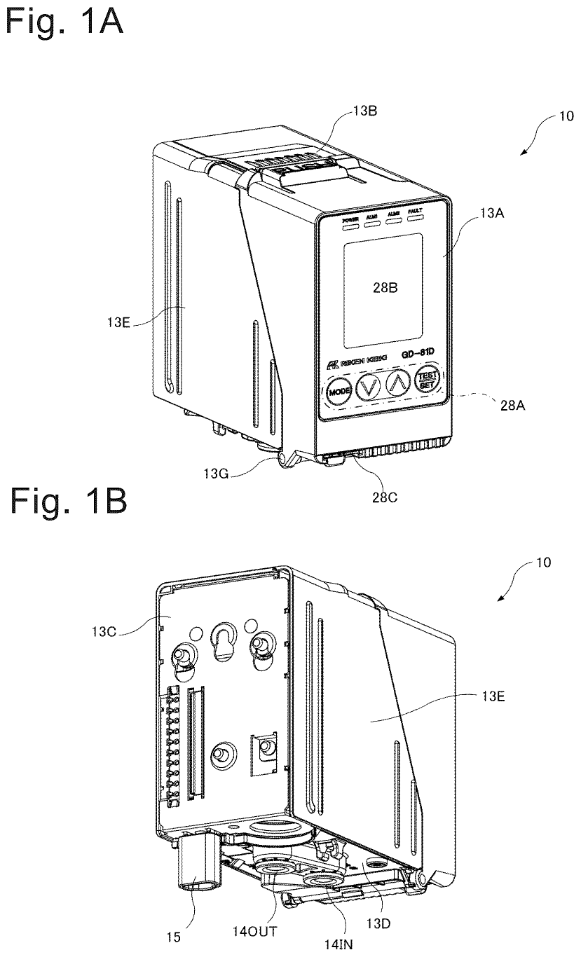

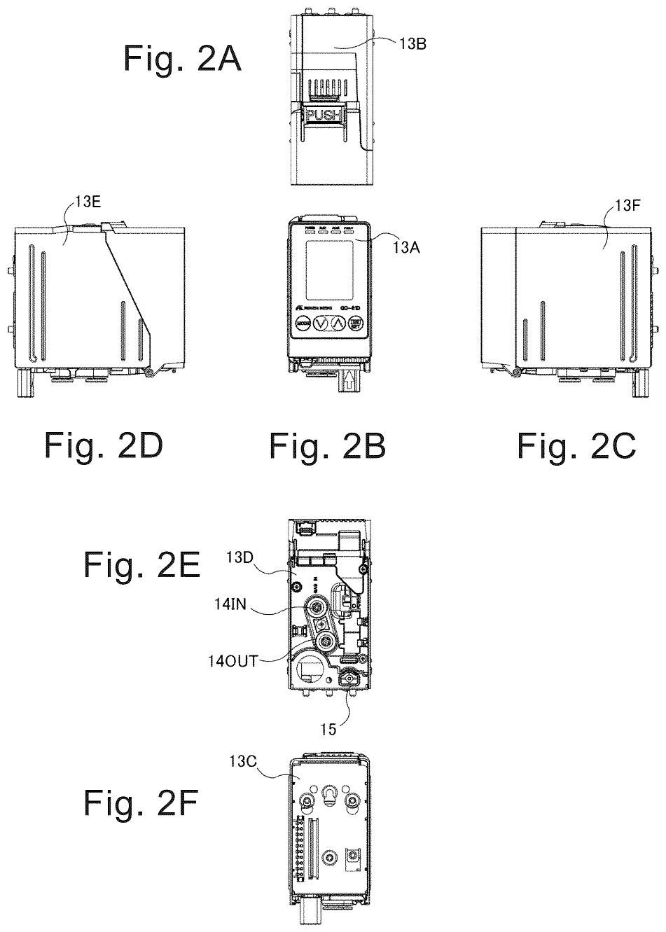

As shown in FIGS. 1A, 1B, and 2A to 2F, the main body 10 includes a front cover 13A, a top cover 13B, a rear cover 13C, a bottom cover 13D, a left side cover 13E, and a right side cover 13F as its housing. In the following description, the direction from the front to the rear in FIGS. 1A and 1B may be referred to as a “depth direction”, the direction from the top to the bottom as a “vertical direction”, and the direction from the right side to the left side as a “width direction”.

Various operation buttons 28A, a display device 28B, and an external communication connector 28C are disposed on the front side of the front cover 13A. A suction port 14IN for taking atmospheric gas into the main body and an exhaust port 14OUT for discharging the gas sucked in through the suction port 14IN are formed in the bottom cover 13D. A lever 15 to be operated in attaching and detaching the gas detector 1 itself to/from a cradle (not shown) is projected downward from the bottom cover 13D.

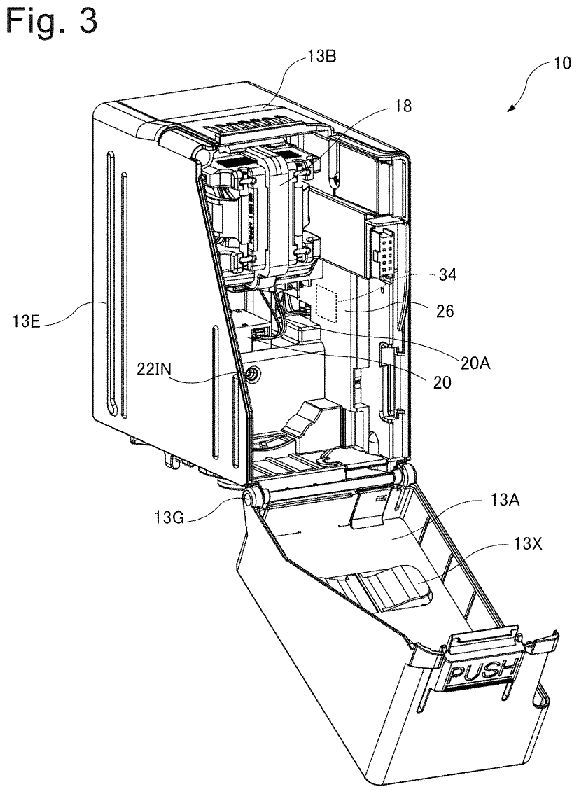

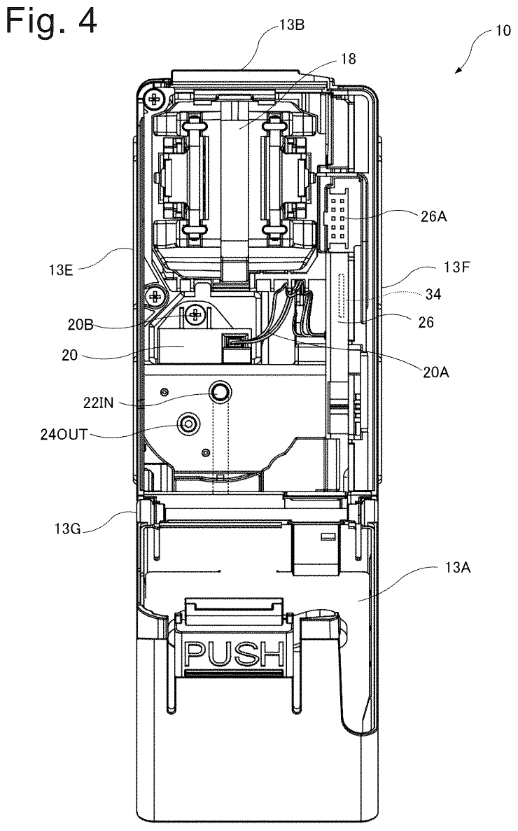

As shown in FIGS. 3 and 4, a hinge 13G with a rotation axis extending in the width direction is located at the border between the bottom edge of the front cover 13A and the front edge of the bottom cover 13D. The entire front cover 13A swings about the hinge 13G to the near side in the depth direction and downward in the vertical direction. As a result, the internal space of the housing of the main body 10 is fully opened at the front side. An elastic body 13X for pressing the gas detection module 50 to the far side when the front cover 13A is closed is disposed on the inner surface of the front cover 13A.

### (Internal Structure of Main Body)

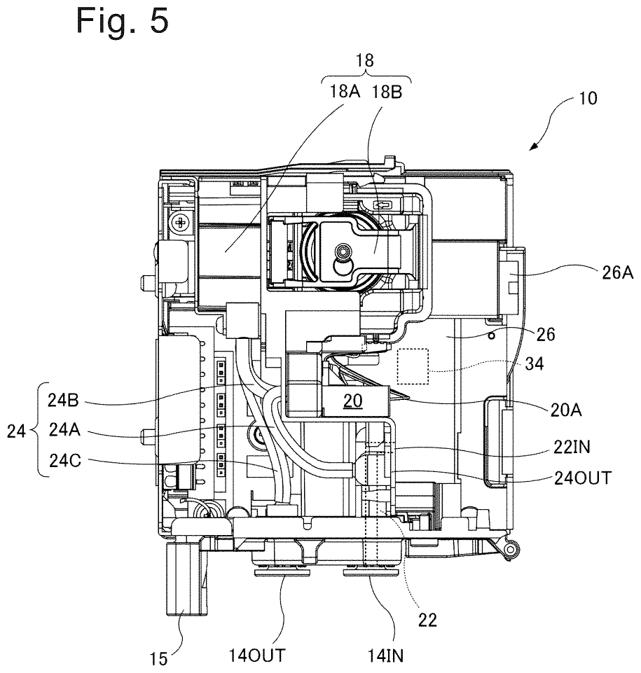

As shown in FIG. 5 where the left side cover 13E and the front cover 13A are omitted, a pump 18, a flow sensor 20, a main body-side inflow path 22, a main body-side outflow path 24, a main body-side circuit substrate 26 (accommodated in a case), a main body-side inlet 22IN, and a main body-side outlet 24OUT are disposed inside the main body 10. As employed herein, the “inflow” of the main body-side inflow path 22 refers to a flow from the suction port 14IN to a gas detection unit of the gas detection module 50. The “outflow” of the main body-side outflow path 24 refers to a flow from the gas detection unit of the gas detection module 50 to the exhaust port 14OUT. The main body-side inflow path 22 is a gas passage directly connecting the suction port 14IN and the main body-side inlet 22IN. The main body-side outflow path 24 includes a first outflow path 24A connecting the main body-side outlet 24OUT and the flow sensor 20, a second outflow path 24B connecting the flow sensor 20 and the pump 18, and a third outflow path 24C connecting the pump 18 and the exhaust port 14OUT.

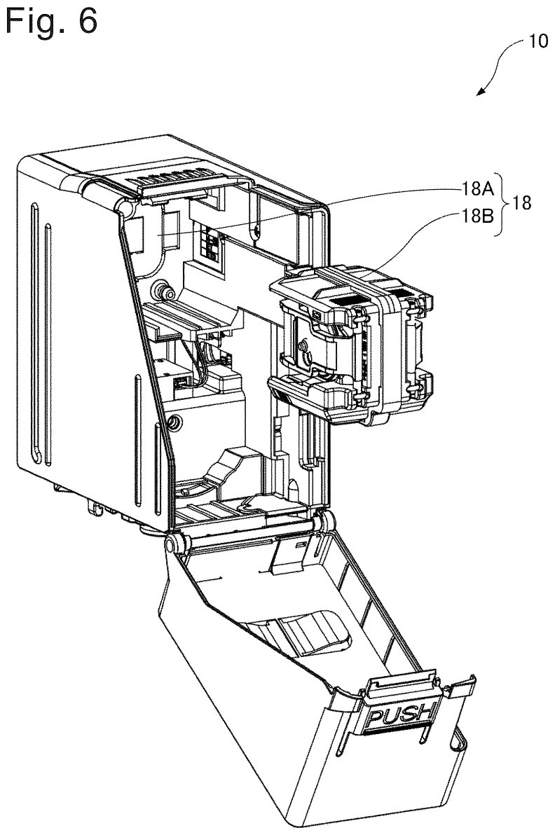

The pump 18 press-feeds the gas through the main body-side outflow path 24 in the outflow direction. The pump 18 includes an electromagnetic coil 18A fixed to the housing, and a diaphragm pump body 18B driven by the electromagnetic coil 18A. The diaphragm pump body 18B is configured to pump out gas by vibrating a permanent magnet fixed to a rod through alternating-current driving of the electromagnetic coil 18A so that a diaphragm fixed to the end of the rod changes the volume of the casing. As shown in FIG. 6, the diaphragm pump body 18B can be easily detached by pulling to the near side. This enables quick replacement of the diaphragm pump body 18B in the event of a failure, by opening the front cover 13A.

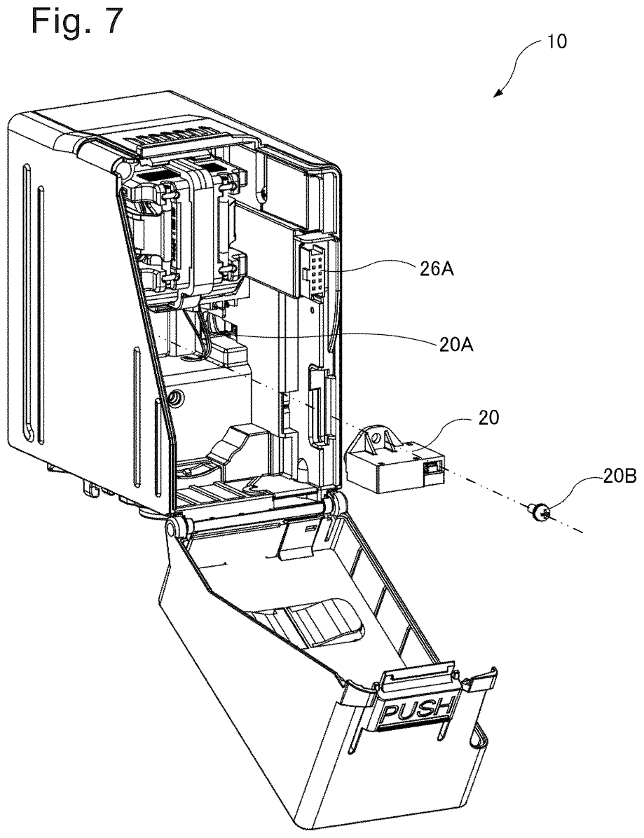

The flow sensor 20 measures the flow rate of the gas flowing through the main body-side outflow path 24. The flow sensor 20 is electrically connected by connector-equipped wiring 20A extending from the main body-side circuit substrate 26. As shown in FIG. 7, the flow sensor 20 is fixed to the housing by a fixing screw 20B extending in the depth direction. With the wiring 20A and the fixing screw 20B detached, the flow sensor 20 can thus be easily detached by pulling to the near side in the depth direction. This enables quick replacement of the flow sensor 20 in the event of a failure, by opening the front cover 13A.

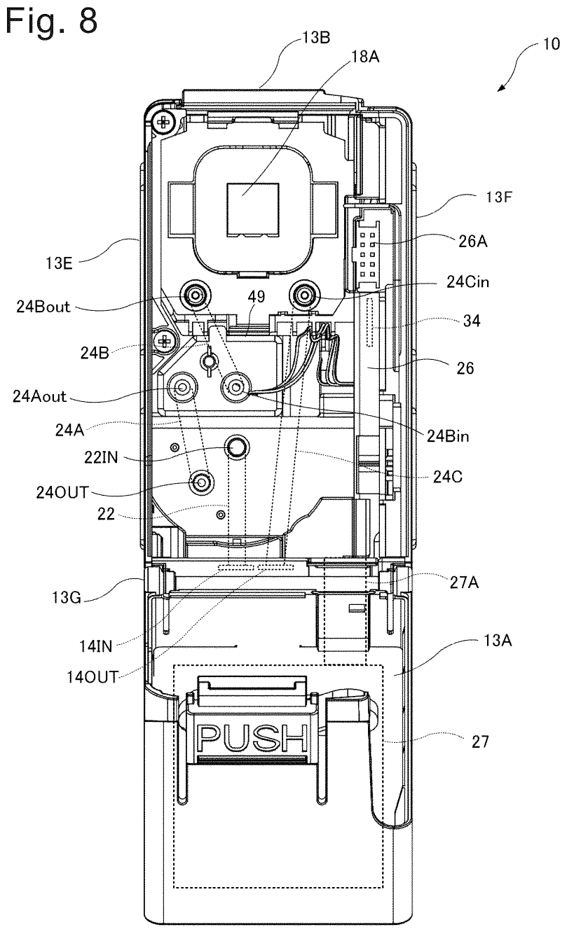

FIG. 8 shows the main body 10 with both the diaphragm pump body 18B and the flow sensor 20 detached. An outlet 24Bout of the second outflow path 24B and an inlet 24Cin of the third outflow path 24C are formed in the main body 10 near the electromagnetic coil 18A. When the diaphragm pump body 18B is pushed in for installation, the outlet 24Bout and the inlet 24Cin are connected to the diaphragm pump body 18B.

An outlet 24Aout of the first outflow path 24A and an inlet 24Bin of the second outflow path 24B are formed in the inner side of the housing where the flow sensor 20 is removed. When the flow sensor 20 is pushed in for installation, the outlet 24Aout and the inlet 24Bin are connected to the flow sensor 20.

The suction port 14IN and the exhaust port 14OUT, the main body-side inlet 22IN and the main body-side outlet 24OUT, the flow sensor 20 (outlet 24Aout and inlet 24Bin), and the pump 18 (outlet 24Bout and inlet 24Cin) are arranged in this order from below to above inside the main body 10 in a vertically overlapping manner. The main body 10 can thus be extremely compactly configured. Moreover, the pump 18 and the flow sensor 20 can be accessed for maintenance from the front side without detaching the main body 10 from the cradle.

As shown in FIG. 8, the main body-side circuit substrate 26 is disposed in parallel with the right side cover 13F along the inner flat surface of the right side cover 13F. The main body-side circuit substrate 26 controls the entire gas detector 1. The main body-side circuit substrate 26 includes a main power supply circuit (not shown), a main body-side computer (CPU) 34, a main body-side memory (not shown), and a main body-side connector 26A. The main body-side connector 26A serves as a part of an interface for establishing connection with the gas detection module 50.

A main body-side auxiliary substrate 27 is disposed inside the front cover 13A, and electrically connected to the main body-side circuit substrate 26 by flexible wiring 27A passing near the hinge 13G. As a result, the main body-side auxiliary substrate 27 serves as a part of the main body-side circuit substrate 26. The main body-side auxiliary substrate 27 is electrically connected to the various operation buttons 28A, the display device 28B, and the external communication connector 28C disposed on the front cover 13A. As shown in FIG. 1A, the external communication connector 28C can be exposed to the outside, with the front cover 13A closed. Since a communication line of an external computer such as a PC can be connected to the external communication connector 28C with the front cover 13A closed, the various operation buttons 28A can be operated and the display on the display device 28B can be checked while communicating with the external computer.

### (Main Body-Side Interface for Gas Detection Module)

As shown in FIG. 4, the layout of a main body-side interface for connecting the main body 10 and the gas detection module 50 is defined by three locations that are the main body-side connector 26A, the main body-side inlet 22IN, and the main body-side outlet 24OUT. The main body-side connector 26A provides an electrical connection function. The main body-side inlet 22IN and the main body-side outlet 24OUT provide a gas connection function.

### <Gas Detection Module>

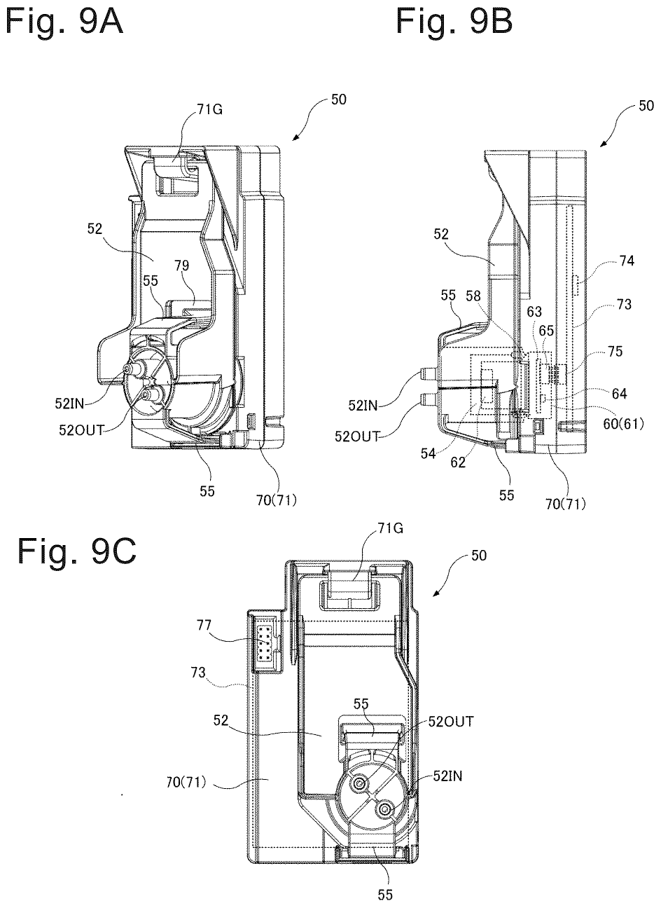

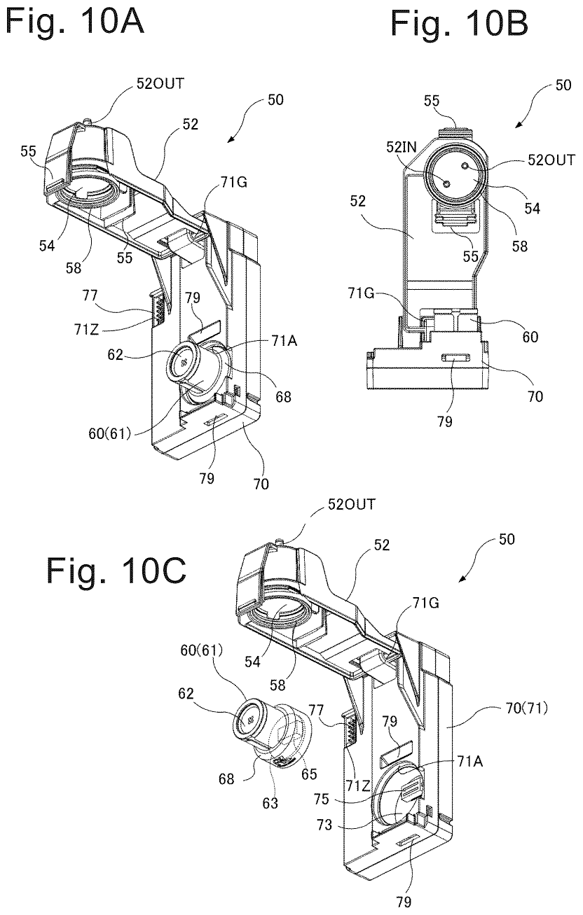

FIGS. 9A to 9C and 10A to 10C show an overall configuration of the gas detection module 50. The gas detection module 50 includes a chamber 52, a gas sensor 60, and a sensor base 70.

### (Chamber)

The chamber 52 includes a chamber-side inflow channel 52IN, a chamber-side outflow channel 52OUT, and an internal space 54. The chamber-side inflow channel 52IN of male structure (protruding structure) is detachably connected to the main body-side inlet 22IN of female structure. The chamber-side outflow channel 52OUT of male structure (protruding structure) is detachably connected to the main body-side outlet 24OUT of female structure. The internal space 54 is interposed between the chamber-side inflow channel 52IN and the chamber-side outflow channel 52OUT. As shown in FIGS. 10A and 10B, the internal space 54 is a cylindrical space with a bottom, and its annular protruding rim serves as a sensor insertion port 58. The chamber-side inflow channel 52IN and the chamber-side outflow channel 52OUT communicate with the bottom of the internal space 54. A rubber seal ring is attached to the sensor insertion port 58. The sensor insertion port 58 (seal ring) is brought into contact with a sealing surface 68 that is an annular tapered surface of the gas sensor 60, whereby the internal space 54 is enclosed. The chamber 52 includes a pair of chamber-side engagement portions 55 constituting an elastic catch structure. The chamber-side engagement portions 55 are engaged with base-side engagement portions 79 that are a pair of receptacle groove structures of the sensor base 70, whereby the chamber 52 and the sensor base 70 are coupled to each other. Since the chamber-side engagement portions 55 have elastic structures, the chamber-side engagement portions 55 and the base-side engagement portions 79 can be disengaged and separated from each other by the user forcefully pulling the chamber 52 and the sensor base 70 apart.

### (Gas Sensor)

As shown in FIG. 9B, the gas sensor 60 includes a sensor housing 61, a gas detection unit 62, a sensor-side circuit substrate 63, a sensor-side computer 64 and a sensor-side memory (not shown) mounted on the sensor-side circuit substrate 63, and a sensor-side connector 65 mounted on the sensor-side circuit substrate 63. The sensor-side circuit substrate 63 provides paths for transmitting electric power and paths for transmitting and receiving signals.

The sensor housing 61 is cylindrical in shape, and the end face located at the protruded end serves as a detection surface of the gas detection unit 62, which comes into contact with the gas in the internal space 54 of the chamber 52. The sealing surface 68 that is an annular tapered surface is formed around the sensor housing 61. The sensor insertion port 58 (seal ring) of the chamber 52 is brought into contact with the sealing surface 68, whereby the internal space 54 at the side of the gas detection unit 62 is enclosed.

The gas detection unit 62 is a part that detects gas using electric power, and can employ various detection methods. Examples of the detection methods that can be employed include the following: a new ceramic method where the amount of heat generated when combustible gas burns on an ultra-fine particle oxidation catalyst (new ceramics) is used for detection; a semiconductor method where a change occurring in resistance when a metal oxide semiconductor is in contact with the detection target gas is detected as the gas concentration; a hot-wire semiconductor method where a change occurring in resistance when a sintered metal oxide semiconductor around a heated noble metal wire coil is in contact with the detection target gas is detected as the gas concentration; a thermal conduction method where a change in thermal conductivity due to the detection target gas is detected as the gas concentration; a constant potential electrolysis method where the detection target gas is electrolyzed on an electrode maintained at a constant potential and the resulting current is detected as the gas concentration; and an infrared method where a measurement cell (space) is irradiated with infrared rays and the amount of change in infrared rays due to absorption by the detection target gas is detected. For example, if the gas detection unit 62 employs the hot-wire semiconductor method, a heater unit including a noble metal wire coil is built in the gas detection unit 62. In other words, a plurality of types of gas sensors 60 with different detection methods are prepared as a series, with the sealing surfaces 68 and the sensor-side connectors 65 of the housings in a common layout.

The sensor-side circuit substrate 63 is a circuit substrate that supplies power to the gas detection unit 62 and outputs the resulting response (analog response or digital response) of the gas detection unit 62. The sensor-side computer 64 converts the analog response (measurement result) of the gas detection unit 62 into a digital signal and outputs the digital signal. The sensor-side computer 64 further normalizes the digital signal and outputs the normalized digital signal. The sensor-side computer 64 also outputs its own type information and various types of setting information stored in the sensor-side memory. The sensor-side computer 64 receives command signals transmitted from the main body-side computer 34 or a base-side computer 74, and generates and outputs response signals based on the commands.

The sensor-side connector 65 is detachably connected to a sensor receptacle connector of the sensor base 70 to be described below. The gas sensor 60 is fixed to the sensor base 70 by the sensor-side connector 65. The gas sensor 60 is supplied with power from outside (sensor base 70), is controlled from outside, and receives commands (signals) from outside via the sensor-side connector 65. The gas sensor 60 outputs analog and digital signals to outside (sensor base 70) via the sensor-side connector 65. As many pins as the number of electrical paths are protruded from the sensor-side connector 65. The pins are inserted into pin holes in the sensor receptacle connector for electrical connection.

### (Sensor Base)

As shown in FIGS. 9B and 9C, the sensor base 70 includes a base housing 71, a base-side circuit substrate 73, the base-side computer 74 and a base-side memory (not shown) mounted on the base-side circuit substrate 73, a sensor receptacle connector 75 mounted on the base-side circuit substrate 73, and a main body receptacle connector 77 mounted on the base-side circuit substrate 73. The base-side circuit substrate 73 provides paths for transmitting power and paths for transmitting and receiving signals.

The base housing 71 is a case of rectangular solid shape, and accommodates the base-side circuit substrate 73 inside. The gas sensor 60 and the chamber 52 are disposed on the rear surface of the base housing 71 (here, the rear surface corresponds to the inner surface when the sensor base 70 is set on the main body 10). As shown in FIGS. 10A to 10C, a circular sensor insertion opening 71A is formed in the base housing 71 at a position corresponding to the sensor receptacle connector 75 of the base-side circuit substrate 73. The sensor insertion opening 71A exposes the entire sensor receptacle connector 75 and a part of the base-side substrate 73 to the outside. The gas sensor 60 is inserted through the sensor insertion opening 71A, whereby the sensor receptacle connector 75 and the sensor-side connector 65 are connected to each other. A rectangular main body connection opening 71Z is formed in the base housing 71 at a position corresponding to the main body receptacle connector 77 of the base-side circuit substrate 73, whereby the main body receptacle connector 77 is exposed. The main body receptacle connector 77 is electrically connected to the main body-side connector 26A.

The base-side engagement portions 79 that are a pair of receptacle groove structures are formed in the base housing 71. The chamber-side engagement portions 55 of the chamber 52 are engaged with the base-side engagement portions 79. The chamber-side engagement portions 55 and the base-side engagement portions 79 can be disengaged and separated from each other by the user forcefully pulling the chamber 52 and the sensor base 70 apart.

A hinge 71G is formed between the base housing 71 and the chamber 52. The chamber 52 and the sensor base 70 swing with respect to each other about the swing shaft of the hinge 71G. When the chamber-side engagement portions 55 and the base-side engagement portions 79 are disengaged, the base housing 71 and the chamber 52 can therefore freely swing with respect to each other via the hinge 71G but remain connected to each other. This can prevent the chamber 52 from being dropped and damaged or lost during the operation of attaching and detaching the gas sensor 60 to/from the base housing 71.

The base-side computer 74 transmits the normalized digital signal received from the gas sensor 60 to the main body-side computer 34. While details will be described in a second embodiment, the base-side computer 74, if a plurality of gas sensors 60 (gas detection units 62) are connected to the gas detection module, multiplexes and converts the digital signals into a sequential signal and transmits the sequential signal to the main body-side computer 34. If the gas sensor 60 is of a type without the built-in sensor-side computer 64, the base-side computer 74 can digitize and normalize the analog signal of the gas detection unit 62 instead of the sensor-side computer 64. The base-side computer 74 can output its type information and various types of setting information stored in the base-side memory to the main body-side computer 34. The base-side computer 74 can receive command signals transmitted from the main body-side computer 34 or the sensor-side computer 64 and output response signals based on the commands.

### (Sensor-Side Interface for Main Body)

As shown in FIG. 9C, the layout of a sensor-side interface for connecting the gas detection module 50 and the main body 10 is defined by three locations that are the main body receptacle connector 77, the chamber-side inflow channel 52IN, and the chamber-side outflow channel 52OUT. In other words, the sensor-side interface perfectly matches the main body-side interface of FIG. 4 (the layout of the main body-side connector 26A, the main body-side inlet 22IN, and the main body-side outlet 24OUT).

### <Connection Between Main Body and Gas Detection Module>

FIGS. 11A and 11B show a state of connection between the main body 10 and the gas detection module 50 in the gas detector 1. Since the main body-side interface (the layout of the main body-side connector 26A, the main body-side inlet 22IN, and the main body-side outlet 24OUT) and the sensor-side interface (the layout of the main body receptacle connector 77, the chamber-side inflow channel 52IN, and the chamber-side outflow channel 52OUT) match perfectly, the main body 10 and the gas detection module 50 are connected to each other by simply pushing the gas detection module 50 into the main body 10 to the far side in the depth direction (see FIG. 11B). Conversely, the main body 10 and the gas detection module 50 are separated from each other by simply pulling the gas detection module 50 out of the main body 10 to the near side in the depth direction (see FIG. 11A).

### <Circuit Configuration>

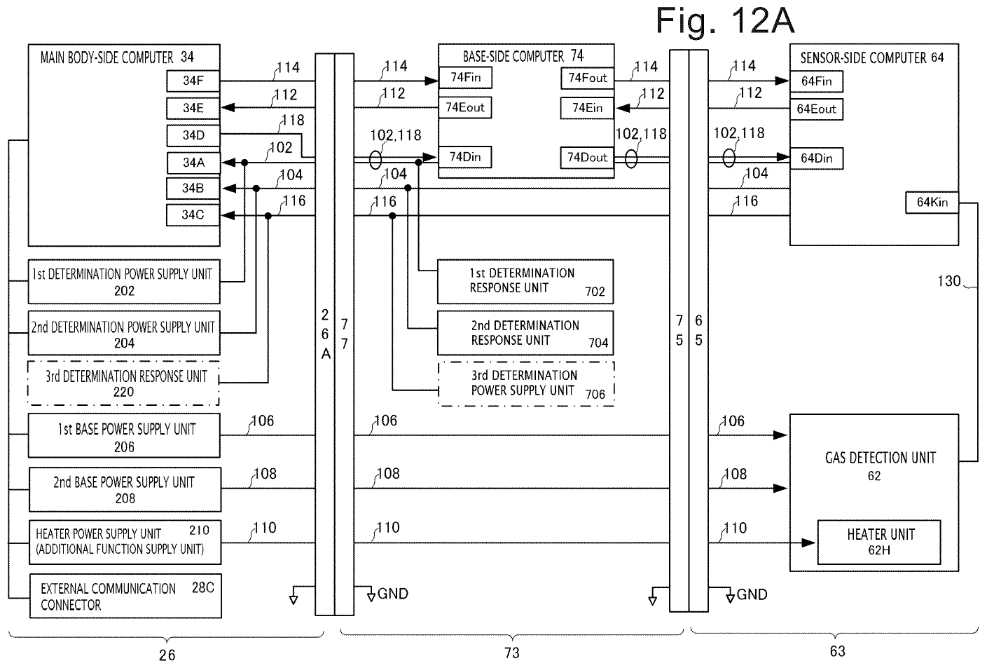

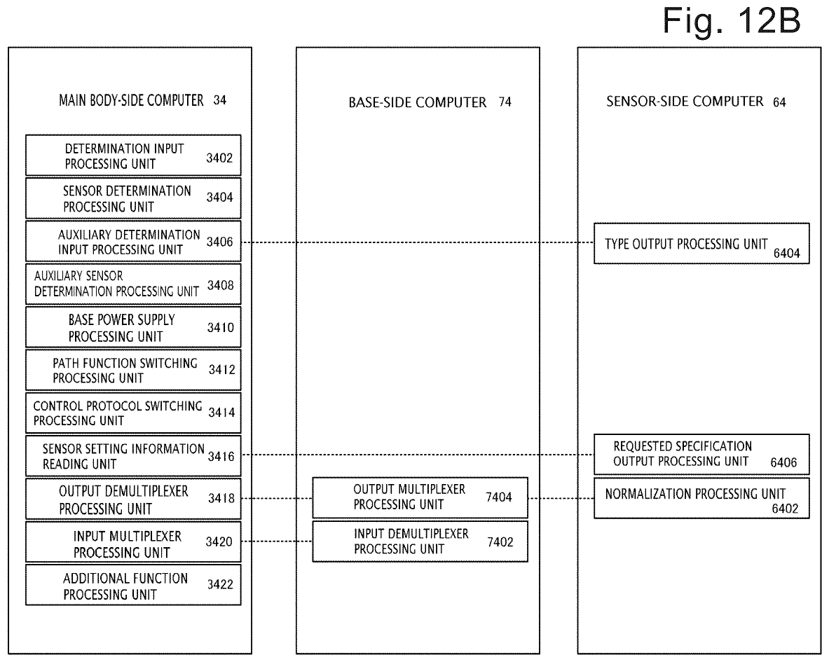

FIG. 12A shows an overall circuit configuration of the gas detector 1.

### (Main Body-Side Circuit Configuration)

The main body-side circuit substrate 26 includes the main body-side computer 34, a first determination power supply unit 202, a second determination power supply unit 204, a first base power supply unit 206, a second base power supply unit 208, a third determination response unit 220, a heater power supply unit (additional function supply unit) 210, the external communication connector 28C, and the main body-side connector 26A.

The main body-side circuit substrate 26 includes a first determination path 102, a second determination path 104, a third determination path 116, a first base power path 106, a second base power path 108, a heater power path 110, a first communication path 112, a second communication path 118, and a reset path 114 as electrical paths connected to the gas detection module 50 via the main body-side connector 26A. The first determination path 102 and the second communication path 118 are implemented by a single path.

The first determination power supply unit 202 includes a 3.3-V power supply and a first main body-side resistor connected thereto in series, for example. The first determination power supply unit 202 supplies first determination power to the gas detection module 50 via the first determination path 102. The second determination power supply unit 204 includes a 3.3-V power supply and a second main body-side resistor connected thereto in series, for example. The second determination power supply unit 204 supplies second determination power to the gas detection module 50 via the second determination path 104. The first base power supply unit 206 is a 5.0-V power supply, for example. The first base power supply unit 206 supplies first base power to the gas detection module 50 via the first base power path 106. The second base power supply unit 208 is a 24-V power supply, for example. The second base power supply unit 208 supplies second base power to the gas detection module 50 via the second base power path 108. The heater power supply unit (additional function supply unit) 210 is a variable voltage power supply, for example. The heater power supply unit 210 supplies heater power, which is adjustable (controlled) within the range of 1.0 to 10.0 V by the main body 10, to the gas detection module 50 via the heater power path 110.

For example, the maximum supply voltages of the first and second determination power supply units 202 and 204 are set to be lower than or equal to, or desirably lower than, the minimum supply voltages of the first and second base power supply units 206 and 208. The reason is to prevent the determination power supplied before the determination of the gas detection module 50 from exceeding the allowable voltage of the gas detection module 50 and causing a failure.

The third determination response unit 220 is a third main body-side resistor connected between the third determination path 116 and the ground, for example, and manages the voltage level of the third determination path 116. For example, the voltage level of the third determination path 116 is the divided voltage between a third sensor-side resistor of a third determination power supply unit 706 to be described below and the third main body-side resistor. In other words, the voltage level of the third determination path 116 can be changed by changing the voltage of the third determination power supply unit 706 and/or the resistance of the third sensor-side resistor.

The main body-side computer 34 includes a first determination unit 34A that detects the voltage level of the first determination path 102, a second determination unit 34B that detects the voltage level of the second determination path 104, and a third determination unit 34C that detects the voltage level of the third determination path 116. The main body-side computer 34 determines the type of the gas detection module 50 on the basis of the voltage levels detected by the first to third determination units 34A to 34C.

The second communication path 118 transmits (outputs) a command signal generated by the main body-side computer 34 to the gas detection module 50. The second communication path 118 is thus connected to a command transmission unit 34D of the main body-side computer 34. The second communication path 118 and the first determination path 102 are merged into a single path within the main body-side circuit substrate 26 and connected to the main body-side connector 26A. This reduces the number of pins (number of paths) of the main body-side connector 26A. In the present embodiment, the number of pins of the main body-side connector 26A excluding ground pins GND is less than or equal to 10 (specifically, eight). In operating the second communication path 118, the main body-side computer 34 can switch a first determination power general-purpose input setting of the first determination unit 34A to a communication output setting of the command transmission unit 34D.

The main body-side computer 34 receives (inputs) the digital signal transmitted from the gas detection module 50 through the first communication path 112. The first communication path 112 is thus connected to a signal reception unit 34E of the main body-side computer 34.

The reset path 114 transmits a reset signal generated by the main body-side computer 34 to the gas detection module 50. The reset path 114 is thus connected to a reset transmission unit 34F of the main body-side computer 34. When the reset signal is deactivated, the gas detection module 50 starts gas detection.

### (Base-Side Circuit Configuration)

The base-side circuit substrate 73 includes the base-side computer 74, the sensor receptacle connector 75, the main body receptacle connector 77, a first determination response unit 702, a second determination response unit 704, and a third determination power supply unit 706.

The base-side circuit substrate 73 includes a first determination path 102, a second determination path 104, a third determination path 116, a first base power path 106, a second base power path 108, a heater power path 110, a first communication path 112, a reset path 114, and a second communication path 118 as paths for electrically connecting the main body receptacle connector 77 and the sensor receptacle connector 75. The first determination path 102 and the second communication path 118 share a single path. The base-side computer 74 is interposed midway along the first communication path 112, the reset path 114, and the second communication path 118.

The first determination response unit 702 is a first response-side resistor connected between the first determination path 102 and the ground, for example. The first determination response unit 702 sets the voltage level of the first determination path 102. For example, when the first determination power supply unit 202 supplies a voltage of 3.3 V, the voltage level of the first determination path 102 is the divided voltage between the first main body-side resistor in the first determination power supply unit 202 and the first response-side resistor. In other words, if the value of the first main body-side resistor is fixed, the voltage level of the first determination path 102 can be uniquely determined by using the value of the first response-side resistor. This voltage level is detected by the first determination unit 34A of the main body-side computer 34. If the value of the first response-side resistor of the first determination response unit 702 is set to a value different from those of the first response-side resistors of the first determination response units in gas detection modules according to second and third embodiments to be described below, the voltage level detected by the main body-side computer 34 therefore also differs. The type of the gas detection module 50 and/or the type of the sensor base 70 can thus be determined.

The second determination response unit 704 is a second response-side resistor connected between the second determination path 104 and the ground, for example. The second determination response unit 704 sets the voltage level of the second determination path 104. For example, when the second determination power supply unit 204 supplies a voltage of 3.3 V, the voltage level of the second determination path 104 is the divided voltage between the second main body-side resistor in the second determination power supply unit 204 and the second response-side resistor. In other words, if the value of the second main body-side resistor is fixed, the voltage level of the second determination path 104 can be uniquely determined by using the value of the second response-side resistor. This voltage level is detected by the second determination unit 34B of the main body-side computer 34. If the value of the second response-side resistor of the second determination response unit 704 is set to a value different from those of the second response-side resistors of the second determination response units in the gas detection modules according to the second and third embodiments to be described below, the voltage level detected by the main body-side computer 34 therefore also differs. The type of the gas detection module 50 and/or the type of the sensor base 70 can thus be determined.

The main body-side computer 34 can determine a wider variety of types of gas detection modules by combining the voltage level of the first determination path 102 and the voltage level of the second determination path 104 (details will be described below).

The third determination power supply unit 706 includes a 3.3-V power supply using a regulator (power supply IC) or the like, and a third sensor-side resistor connected thereto in series, for example. The voltage level of the third determination path 116 is the divided voltage between the third sensor-side resistor and the third main body-side resistor in the third determination response unit 220 of the main body 10, for example. The voltage level of the third determination path 116 is detected by the third determination unit 34C of the main body-side computer 34. The voltage level of the third determination path 116 can be used in determining a special gas detection module that is unable to be determined by combining the voltage levels of the first and second determination paths 102 and 104.

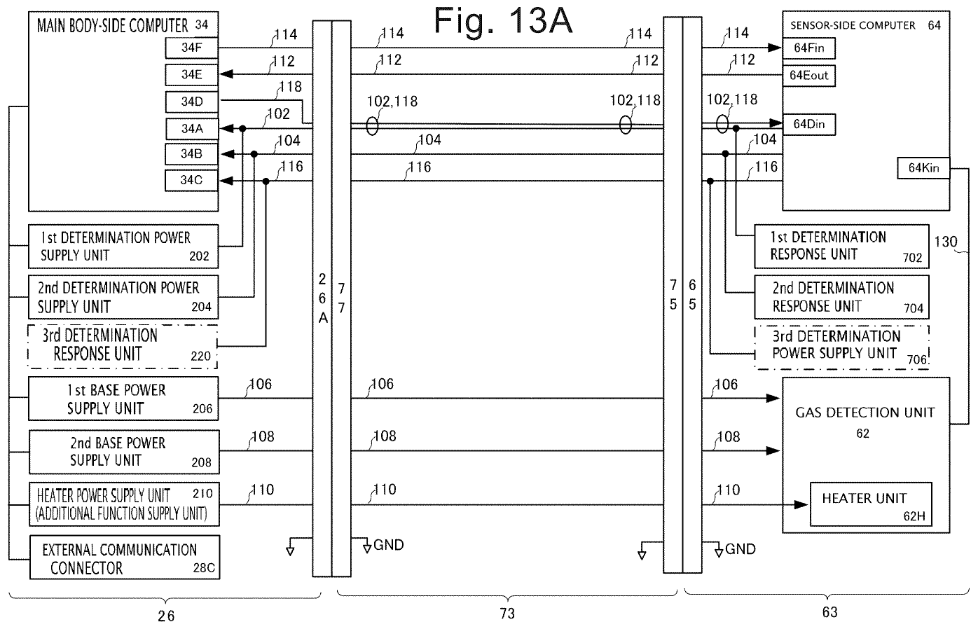

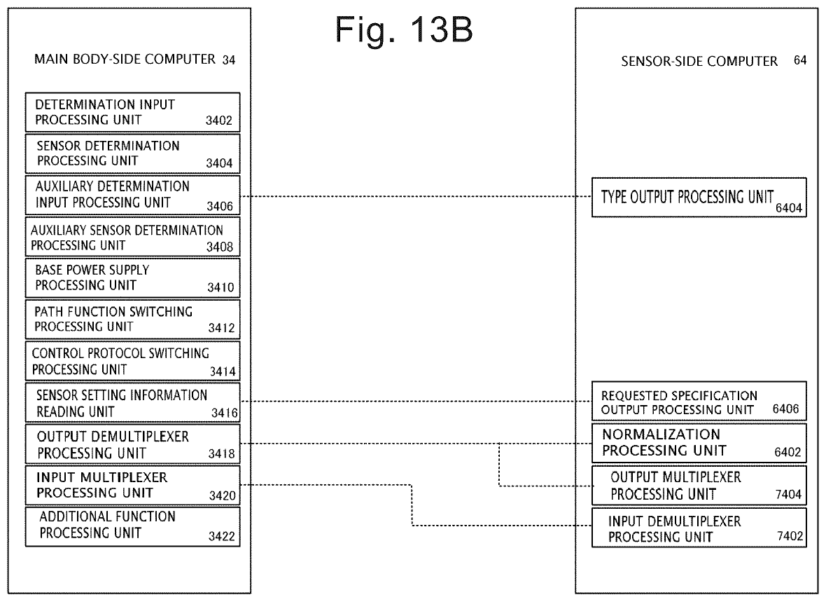

In the present embodiment, the entire first and second determination response units 702 and 704 are described to be disposed on the base-side circuit substrate 73. However, the present invention is not limited thereto. For example, a part or all of the first and second determination response units 702 and 704 may be disposed on the sensor-side circuit substrate 63 (see FIG. 13A). Such a configuration, combined with changing the values of first and third response-side resistors for respective different types of gas sensors 60, enables the main body-side computer 34 to determine the type of gas sensor 60 even if the gas sensor 60 is replaced with another type without replacing the sensor base 70. The second communication path 118 extending from the main body receptacle connector 77 is connected to a command reception unit 74Din of the base-side computer 74. Commands received by the command reception unit 74Din are output to the second communication path 118 connected to a command transmission unit 74Dout of the base-side computer 74 and transmitted to the sensor receptacle connector 75.

Meanwhile, the first communication path 112 extending from the sensor receptacle connector 75 is connected to a signal reception unit 74Ein of the base-side computer 74. A signal received by the command reception unit 74Ein is subjected to predetermined processing and then output from a command transmission unit 74Eout of the base-side computer 74 to the first communication path 112 and transmitted to the main body receptacle connector 77.

The reset path 114 extending from the main body receptacle connector 77 is connected to a reset reception unit 74Fin of the base-side computer 74. Commands received by the reset reception unit 74Fin are output to the reset path 114 connected to a reset transmission unit 74Fout of the base-side computer 74 and transmitted to the sensor receptacle connector 75.

### (Sensor-Side Circuit Configuration)

The sensor-side circuit substrate 63 includes the sensor-side computer 64 and the sensor-side connector 65. The gas detection unit 62 is further connected to the sensor-side circuit substrate 63. Depending on the gas detection method, a heater unit 62H such as a noble metal coil can be built in the gas detection unit 62. The sensor-side circuit substrate 63 includes a first determination path 102, a second determination path 104, a third determination path 116, a first base power path 106, a second base power path 108, a heater power path 110, a first communication path 112, a reset path 114, and a second communication path 118 as electrical paths connected to the sensor base 70 via the sensor-side connector 65. The first determination path 102 and the second communication path 118 share a single path.

The second communication path 118 extending from the sensor-side connector 65 is connected to a command reception unit 64Din of the sensor-side computer 64. The sensor-side computer 64 outputs desired responses based on the commands received by the command reception unit 64Din from a signal transmission unit 64Eout. The signal transmission unit 64Eout of the sensor-side computer 64 is connected to the first communication path 112. This first communication path 112 extends to the sensor-side connector 65.

The reset path 114 extending from the sensor-side connector 65 is connected to a reset reception unit 64Fin of the sensor-side computer 64. By referring to commands received by the reset reception unit 64Fin, the sensor-side computer 64 determines whether to output a signal from the signal transmission unit 64Eout (whether to start communication).

The analog output of the gas detection unit 62 is transmitted to an analog reception unit 64Kin of the sensor-side computer 64 through an analog output path 130. The sensor-side computer 64 converts the analog signal received by the analog reception unit 64Kin into a digital signal, normalizes the digital signal, and then outputs the normalized digital signal from the signal transmission unit 64Eout.

The first base power path 106 extending from the sensor-side connector 65 is connected to the gas detection unit 62. The first base power is thereby supplied to the gas detection unit 62. The second base power path 108 extending from the sensor-side connector 65 is connected to the gas detection unit 62. The second base power is thereby supplied to the gas detection unit 62. The heater power path 110 extending from the sensor-side connector 65 is connected to the heater unit 62H. The controlled heater power is thereby supplied to the heater unit 62H. The first base power and the second base power do not need to be simultaneously supplied, and suitable power between the first base power and the second base power may be selectively supplied depending on the type of the gas sensor 60. In such a case, either one of the first and second base power supply paths 106 and 108 may be omitted from the base-side circuit substrate 73 and/or the sensor-side circuit substrate 63. If the gas detection unit 62 does not include the built-in heater unit 62H, the heater power does not need to be supplied. In such a case, the heater power path 110 can be omitted from the base-side circuit substrate 73 and/or the sensor-side circuit substrate 63.

### <Gas Detection Program>

Next, a gas detection program (gas detection procedure) to be performed by the gas detector 1 will be described. This gas detection program is executed by the main body-side computer 34, the base-side computer 74, and the sensor-side computer 64.

FIG. 12B shows functions that the main body-side computer 34, the base-side computer 74, and the sensor-side computer 64 implement by executing the gas detection program (gas detection procedure).

### (Functions of Main Body-Side Computer)

The main body-side computer 34 includes a determination input processing unit 3402, a sensor determination processing unit 3404, an auxiliary determination input processing unit 3406, an auxiliary sensor determination processing unit 3408, a base power supply processing unit 3410, a path function switching processing unit 3412, a control protocol switching processing unit 3414, a sensor setting information reading unit 3416, an output demultiplexer processing unit 3418, an input multiplexer processing unit 3420, and an additional function processing unit 3422.

The determination input processing unit 3402 controls the first determination power supply unit 202 and the second determination power supply unit 204 to supply the first determination power and the second determination power to the first determination path 102 and the second determination path 104 after the main power supply of the main body 10 is turned ON.

The sensor determination processing unit 3404 determines the gas detection module connected to the main body 10 on the basis of the voltage levels of the first determination path 102 and the second determination path 104 to which the first determination power and the second determination power are supplied. The voltage level of the third determination path 116 may be added to the determination conditions.

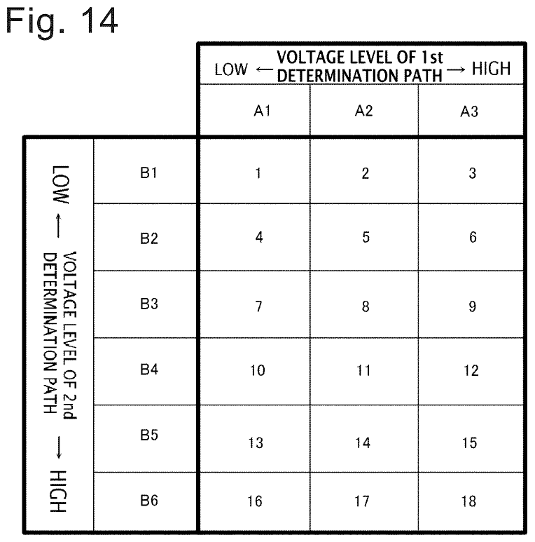

For example, the sensor determination processing unit 3404 determines the gas determination module by referring to a sensor determination table shown in FIG. 14, stored in the main body-side memory. Using this sensor determination table, the sensor determination processing unit 3404 can determine the voltage level of the first determination path 102 in multiple grades (here, three grades A1 to A3) and the voltage level of the second determination path 104 in multiple grades (six grades B1 to B6), and determine a total of 18 types of gas detection modules (sensor types) on the basis of the combinations of the grades. A greater number of types can be determined by combining the voltage level of the third determination path 116.

In the present embodiment, the determination input processing unit 3402 is described to determine the gas detection module on the basis of the voltage levels of the supplied power. However, the present invention is not limited thereto. The sensor determination processing unit 3404 may determine the type of the gas detection module by the determination input processing unit 3402 transmitting a type information transmission command and the base-side computer 74 and/or the sensor-side computer 64 returning sensor type information (response value) stored in the memories.

The auxiliary determination input processing unit 3406 supplies an auxiliary command or auxiliary power (hereinafter, auxiliary determination input) for determining detailed specifications of the gas detection module in an auxiliary manner after the completion of the determination of the gas detection module (sensor base 70 in particular) by the sensor determination processing unit 3404. In particular, the auxiliary determination input processing unit 3406 supplies the auxiliary determination input to determine not the type of the entire gas detection module 50 but the type of the gas sensor 60 mounted thereon. In the present embodiment, the communication functions are assumed to be ON before the auxiliary determination input processing unit 3406 is activated. The auxiliary determination input processing unit 3406 thus transmits the auxiliary command to the base-side computer 74 and/or the sensor-side computer 64 using the second communication path 118. The auxiliary sensor determination processing unit 3408 determines the type of the gas sensor 60 by referring to the response value (auxiliary response value) transmitted from the base-side computer 74 and/or the sensor-side computer 64 (here, the sensor-side computer 64 of the gas sensor 60).

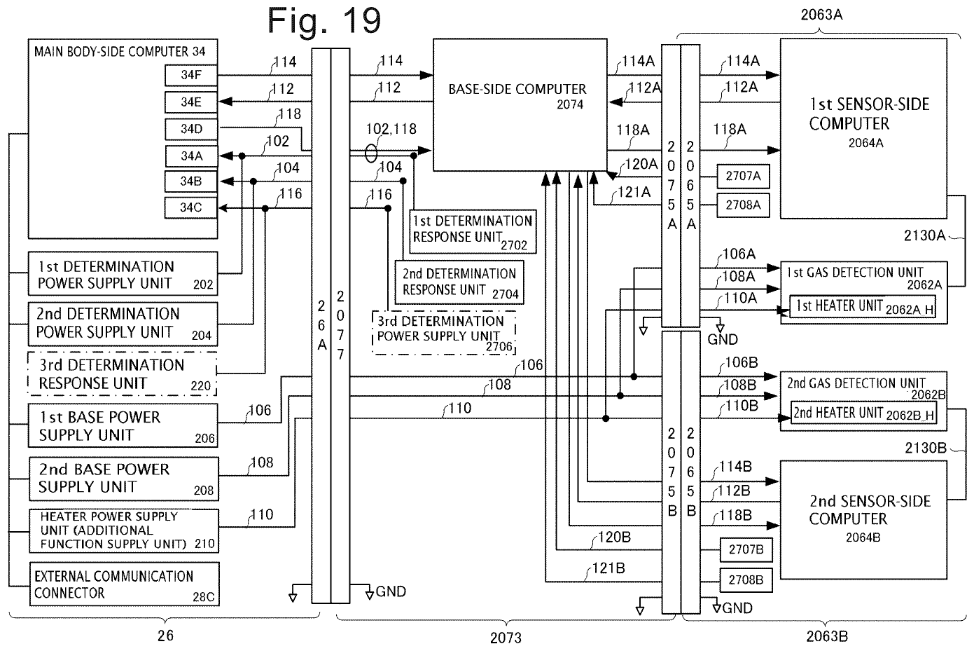

For example, in the case of a gas detection module on which a plurality of gas sensors are mounted as will be described below in the second embodiment with reference to FIG. 19, both the entire module (sensor base) and each of the plurality of gas sensors mounted thereon need to be determined. Here, the entire gas detection module is desirably determined before the start of the communication functions in the first place. In the present embodiment, the entire gas detection module is therefore determined from the voltage levels of the first and second determination paths 102 and 104 using the determination input processing unit 3402 and the sensor determination processing unit 3404. Once the determination of the entire module (sensor base) is completed, the communication function (communication protocol) suited to the gas detection module can be started between the main body-side computer 34 and the base-side computer 74. The auxiliary determination input processing unit 3406 and the auxiliary sensor determination processing unit 3408 then determine details of the gas sensors through signal exchange using command signals and auxiliary response signals.

In the case of the gas detection module shown in FIG. 19, communications between the base-side computer and the sensor-side computers are not guaranteed, since a gas sensor may not be mounted or may malfunction. As shown in FIG. 19, a base-side computer 2074 accepting an auxiliary command from the main body-side computer 34 then supplies determination power to a first sensor-side first determination response unit 2707A and a first sensor-side second determination response unit 2708A of a first sensor-side circuit substrate 2063A and a second sensor-side first determination response unit 2707B and a second sensor-side second determination response unit 2708B of a second sensor-side circuit substrate 2063B, for example. The base-side computer 2074 can detect the response voltage levels, determine the respective gas sensors, and transmit the determination results to the main body-side computer 34 via the first communication path 112. This enables determination of the gas sensors independent of whether the sensor-side computers are activated.

The base power supply processing unit 3410 supplies the base power to the gas detection module 50. Specifically, the base power supply processing unit 3410 supplies appropriate base power to the gas detection module 50 on the basis of the result of determination of the gas detection module 50 by the sensor determination processing unit 3404. Here, the base power supply processing unit 3410 may control the voltage level of the base power, or selectively supply appropriate one of a plurality of base power supplies (first base power supply and second base power supply).

The path function switching processing unit 3412 switches between a mode (sensor determination mode) where the first determination power is supplied to the first determination path 102 using the first determination power supply unit 202 to enable sensor determination through the first determination path 102 and a mode (signal transmission mode) where the first determination power general-purpose output setting is changed to the communication setting to enable signal transmission through the second communication path 118. Since the first determination path 102 and the second communication path 118 can thereby share the single path, the number of connector pins can be reduced.

The control protocol switching processing unit 3414 switches the control protocol (control procedure) for controlling the gas detection module 50 on the basis of the result of determination of the gas detection module 50 by the determination input processing unit 3402 and the sensor determination processing unit 3404 and/or the result of determination by the auxiliary sensor determination processing unit 3408. This enables sensor controls tailored to various gas detection methods that the gas detection unit 62 can employ.

The input multiplexer processing unit 3420 multiplexes a plurality of command signals generated by the main body-side computer 34 into sequential commands. The sequence commands are transmitted to the gas detection module 50 via the second communication path 118. Aside from multiplexing a plurality of command signals for a single gas sensor 60, the multiplexing used herein also includes multiplexing commands for a plurality of gas sensors as with a gas detection module 2050 according to the second embodiment.

The output demultiplexer processing unit 3418 receives a multiplexed sequential output transmitted from the base-side computer 74 and divides the sequential output into individual outputs. The output demultiplexer processing unit 3418 is suitably used in receiving a sequential output into which the measurement results (outputs) of a plurality of gas sensors are multiplexed as with the gas detection module 2050 according to the second embodiment.

The additional function processing unit 3422 determines the supply level (including whether to supply) of the additional function to the gas detection module 50 on the basis of the determination result of the sensor determination processing unit 3404 and/or the determination result of the auxiliary sensor determination processing unit 3408. The additional function processing unit 3422 may control the supply level of the additional function by referring to the sensor determination table shown in FIG. 14. Specifically, the additional function processing unit 3422 controls the heater power supply unit 210 to supply desired controlled power to the gas detection module 50. If the gas detection module 50 or the gas sensor 60 is reduced in size, the heater power supply and the power control function thereof can be unable to be built in. In the present embodiment, the heater power supply unit 210 is thus included in the main body 10 outside the gas detection module 50, and supplies controlled power (additional power) to the gas detection module 50. Since the heater power supply unit 210 remains in the main body 10 in replacing the gas detection module 50 or the gas sensor 60 due to a failure or other reasons, the number of parts to be replaced can be reduced to lower the maintenance cost.

The sensor setting information reading unit 3416 receives requested specification information transmitted from a requested specification output processing unit 6406 included in the sensor-side computer 64 to be described below. The main body-side computer 34 controls the voltage level of the base power and the voltage level of the heater power on the basis of the requested specification information.

### (Functions of Base-Side Computer)

The base-side computer 74 includes an input demultiplexer processing unit 7402 and an output multiplexer processing unit 7404. The input demultiplexer processing unit 7402 divides the multiplexed sequential commands transmitted from the input multiplexer processing unit 3420 of the main body-side computer 34 into individual commands. If a plurality of gas sensors are mounted as with the gas detection module 2050 according to the second embodiment, the multiplexed commands are divided and transmitted to the respective gas sensors. The output multiplexer processing unit 7404 multiplexes the signals output from a plurality of gas sensors as with the gas detection module 2050 according to the second embodiment into a sequential output, and transmits the sequential output to the main body-side computer 34.

### (Functions of Sensor-Side Computer)

The sensor-side computer 64 includes a normalization processing unit 6402, a type output processing unit 6404, and the requested specification output processing unit 6406. The normalization processing unit 6402 digitizes the analog output obtained from the gas sensor 60, and normalizes the resulting digital output according to an output rule (output specification) set by the main body-side computer 34 in advance. This normalization processing unit 6402 enables the main body-side computer 34 and the base-side computer 74 to process the output data without performing special processing even if various types of gas sensors 60 are replaced.

The type output processing unit 6404 outputs type information (for example, information about the gas detection method) stored in the sensor-side memory (not shown) mounted on the sensor-side circuit substrate 63 to the main body-side computer 34 as a response based on the auxiliary command transmitted from the auxiliary determination input processing unit 3406 of the main body-side computer 34. The auxiliary sensor determination processing unit 3408 of the main body-side computer 34 can thereby determine the type of the gas sensor 60.

The requested specification output processing unit 6406 outputs the requested specification information (for example, information about the base voltage level, whether the heater power is needed, and the level of the heater power) stored in the sensor-side memory (not shown) mounted on the sensor-side circuit substrate 63 to the main body-side computer 34 as a response to the command transmitted from the sensor setting information reading unit 3416 of the main body-side computer 34. The sensor setting information reading unit 3416 of the main body-side computer 34 can thereby obtain the setting information about the gas sensor 60.

(Procedure from Main Power Supply ON to Start of Measurement)

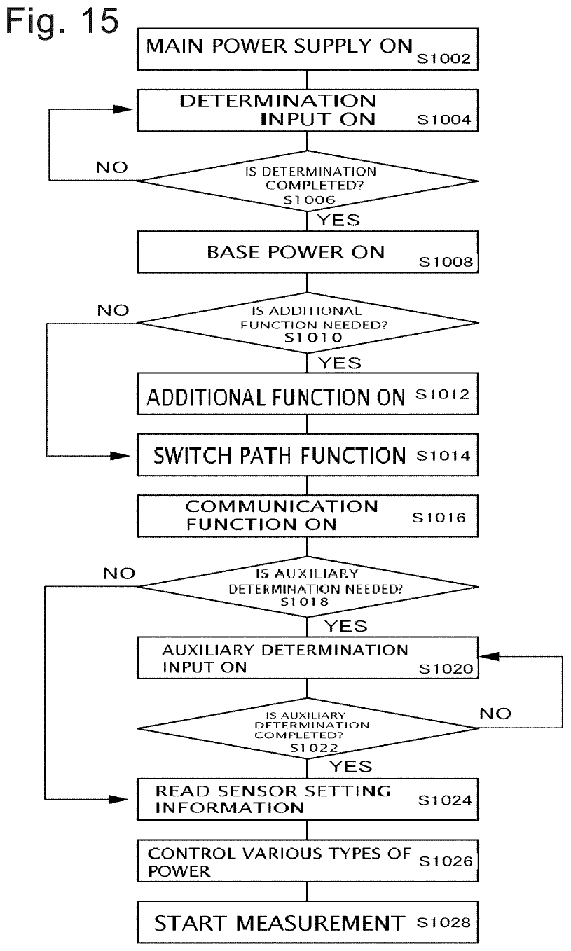

A procedure performed by the gas detector 1 will be described with reference to the flowchart of FIG. 15. Initially, in step S1002, the main power supply of the gas detector 1 is turned ON. In step S1004, a determination input becomes ON. This “determination input ON” means that the first determination power supply unit 202 and the second determination power supply unit 204 start to supply determination power. In step S1006, whether the gas detection module 50 is successfully determined is determined. If the gas detection module 50 is not successfully determined (NO), one of the following cases applies: the gas detection module 50 is not connected, the gas detection module 50 is malfunctioning, and an unidentifiable gas detection module is connected. The processing thus returns to step S1004, and the gas detector 1 waits with the determination input ON. If the gas detection module 50 is successfully determined in step S1006 (YES), the processing proceeds to step S1008, and the base power suited to the determined type is supplied to the gas detection module 50.

Next, the processing proceeds to step S1010, and whether the determined type needs the additional function (for example, heater function) is determined. If the additional function is needed (YES), the processing proceeds to step S1012. The additional function (for example, heater power supply) is turned ON, and the processing proceeds to step S1014. On the other hand, if the additional function is not needed (NO), the processing skips step S1012 and proceeds to step S1014. In step S1014, the main body-side computer 34 switches the first determination path 102 to the second communication path 118 by changing the setting of the power supply by the first determination power supply unit 202 in step S1004 from the general-purpose input state for the first determination unit 34A to detect to the communication output state of the communication transmission unit 34D. The gas detector 1 thereby becomes ready for communication.

Next, the processing proceeds to step S1016. The reset signal generated by the main body-side computer 34 is deactivated, whereby the signal communication between the main body-side computer 34, the base-side computer 74, and the sensor-side computer 64 is turned ON. Then, in step S1018, whether the type of the gas detection module 50 needs to be determined in more detail (auxiliary determination) is determined. For example, if the gas detection module 50 includes a plurality of gas sensors, auxiliary determination is determined to be needed (YES), and the processing proceeds to step S1020 to turn auxiliary determination input ON. This “auxiliary determination input ON” means that the auxiliary determination input processing unit 3406 of the main body-side computer 34 transmits an auxiliary command. The processing then proceeds to step S1022, and the main body-side computer 34 waits for the sensor-side computer 64 to respond (return type information). If appropriate type information is successfully obtained (YES), the processing proceeds to step S1024. On the other hand, if appropriate type information is not successfully obtained in step S1022 (NO), the processing returns to step S1020 to retransmit an auxiliary command. If, in step S1018, more detailed determination (auxiliary determination) of the type of the gas detection module 50 is determined to not be needed (NO), the processing skips the foregoing steps S1020 and S1022 and proceeds to step S1024.

In step S1024, the main body-side computer 34 obtains sensor setting information (requested specification information) from the sensor-side computer 64. In step S1026, the main body-side computer 34 adjusts the base power, heater power, etc. in detail on the basis of the sensor setting information. Through the foregoing procedure, the gas detector 1 becomes ready for measurement. In step S1028, the gas detector 1 starts gas measurement.

In FIGS. 12A and 12B, the base-side circuit substrate 73 is described to include the base-side computer 74, the first determination response unit 702, the second determination response unit 704, and the third determination power supply unit 706. However, the present invention is not limited thereto. For example, as shown in FIGS. 13A and 13B, the input demultiplexer processing unit 7402 and the output multiplexer processing unit 7404 that are the functions of the base-side computer 74 may be included in the sensor-side computer 64, and the base-side computer 74 may be omitted. Similarly, the first determination response unit 702, the second determination response unit 704, and the third determination power supply unit 706 may be disposed on the sensor-side circuit substrate 63. In such a configuration, the base-side circuit substrate 73 may be configured as a relay substrate between the main body-side connector 26A and the sensor-side connector 65. If the main body-side connector 26A and the sensor-side connector 65 have different numbers of pins, the base-side circuit substrate 73 can absorb the difference in the number of pins.

### Second Embodiment

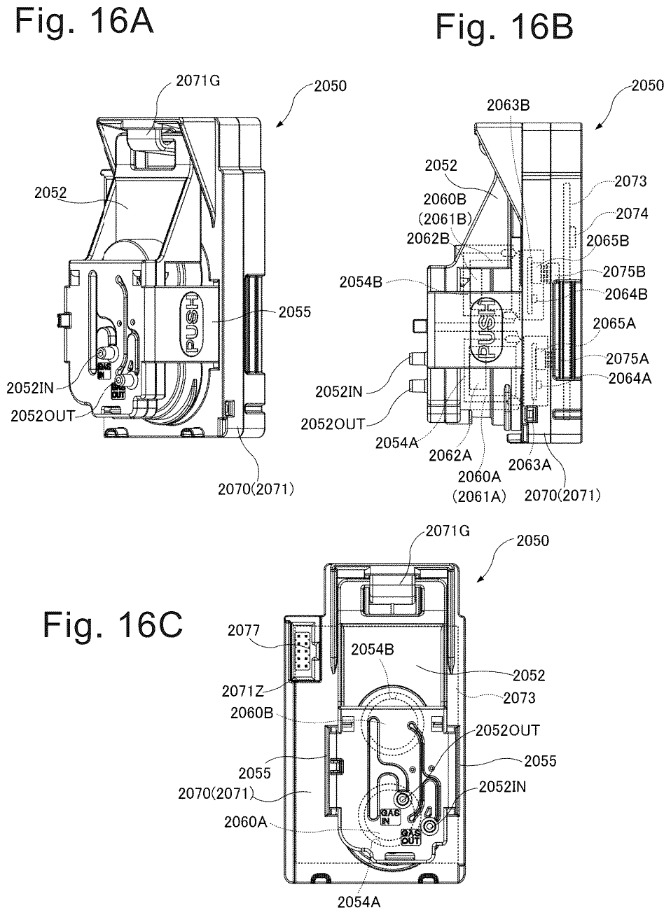

Next, a gas detector 2001 according to the second embodiment will be described with reference to FIGS. 16A to 16C and subsequent figures. A main body 10 of the gas detection unit 2001 is the same as that of the first embodiment. A description thereof will thus be omitted. Differences of a gas detection module 2050 detachably connected to the main body 10 from the gas detection module 50 according to the first embodiment will mainly be described. The same or similar parts and members between the gas detection module 2050 and the gas detection module 50 are denoted by reference numerals with the same lower three digits, and a detailed description thereof will be omitted.

### <Gas Detection Module>

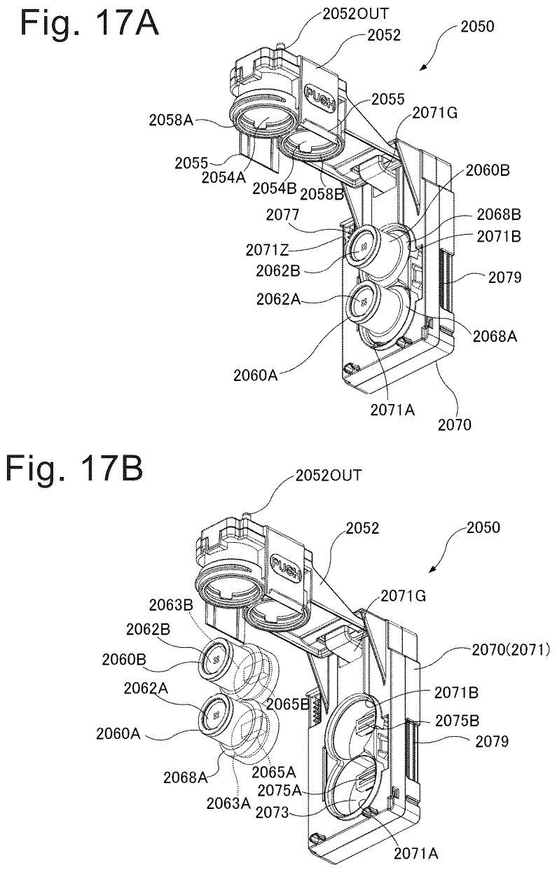

FIGS. 16A to 16C, 17A, and 17B show an overall configuration of the gas detection module 2050. The gas detection module 2050 includes a chamber 2052, a first gas sensor 2060A, a second gas sensor 2060B, and a sensor base 2070.

### (Chamber)

A chamber 2052 includes a chamber-side inflow channel 2052IN, a chamber-side outflow channel 2052OUT, and a first internal space 2054A and a second internal space 2054B. The chamber-side inflow channel 2052IN of male structure (protruding structure) is detachably connected to the main body-side inlet 22IN. The chamber-side outflow channel 2052OUT of male structure (protruding structure) is detachably connected to the main body-side outlet 24OUT. The first and second internal spaces 2054A and 2054B are interposed between the chamber-side inflow channel 2052IN and the chamber-side outflow channel 2052OUT. The first internal space 2054A is a cylindrical space with a bottom, and its annular protruding rim serves as a first sensor insertion port 2058A. The chamber-side inflow channel 2052IN and the chamber-side outflow channel 2052OUT communicate with the bottom of the first internal space 2054A. A rubber seal ring is attached to the first sensor insertion port 2058A. The first sensor insertion port 2058A (seal ring) is brought into contact with a first sealing surface 2068A that is an annular tapered surface of the first gas sensor 2060A, whereby the first internal space 2054A is enclosed.

The second internal space 2054B is vertically juxtaposed with the first internal space 2052A. The second internal space 2054B is a cylindrical space with a bottom, and its annular protruding rim serves as a second sensor insertion port 2058B. The chamber-side inflow channel 2052IN and the chamber-side outflow channel 2052OUT communicate with the bottom of the second internal space 2054B. In other words, each of the chamber-side inflow channel 2052IN and the chamber-side outflow channel 2052OUT is branched and connected to both the first and second internal spaces 2052A and 2054B. A rubber seal ring is attached to the second sensor insertion port 2058B. The second sensor insertion port 2058B (seal ring) is brought into contact with a second sealing surface 2068B that is an annular tapered surface of the second gas sensor 2060B, whereby the second internal space 2054B is enclosed.

The chamber 2052 includes a pair of chamber-side engagement portions 2055 constituting an elastic catch structure. The chamber-side engagement portions 2055 are engaged with base-side engagement portions 2079 that are a pair of receptacle groove structures on the sensor base 2070, whereby the chamber 2052 and the sensor base 2070 are coupled to each other.

### (First Gas Sensor)

As shown in FIG. 16B, the first gas sensor 2060A includes a first sensor housing 2061A, a first gas detection unit 2062A, a first sensor-side circuit substrate 2063A, a first sensor-side computer 2064A and a first sensor-side memory (not shown) mounted on the first sensor-side circuit substrate 2063A, and a first sensor-side connector 2065A mounted on the first sensor-side circuit substrate 2063A. As shown in FIG. 17A, a first sealing surface 2068A is formed around the first sensor housing 2061A. The first sensor insertion port 2058A (seal ring) of the chamber 2052 comes into contact with the first sealing surface 2068A, whereby the first internal space 2054A at the side of the first gas detection unit 2062A is enclosed.

The first sensor-side computer 2064A converts the analog response of the first gas detection unit 2062A into a digital signal and outputs the digital signal. The first sensor-side computer 2064A further normalizes the digital signal of the first gas detection unit 2062A and outputs the normalized digital signal. The first sensor-side computer 2064A also outputs its own first type information and first setting information stored in the first sensor-side memory. The first sensor-side computer 2064A receives command signals transmitted from the main body-side computer 34 or the base-side computer 2074, and outputs response signals based on the commands.

The first sensor-side connector 2065A is detachably connected to a first sensor receptacle connector 2075A of the sensor base 2070. The first gas sensor 2060A is fixed to the sensor base 2070 by the first sensor-side connector 2065A.

### (Second Gas Sensor)

As shown in FIG. 16B, the second gas sensor 2060B includes a second sensor housing 2061B, a second gas detection unit 2062B, a second sensor-side circuit substrate 2063B, a second sensor-side computer 2064B and a second sensor-side memory (not shown) mounted on the second sensor-side circuit substrate 2063B, and a second sensor-side connector 2065B mounted on the second sensor-side circuit substrate 2063B. As shown in FIG. 17A, a second sealing surface 2068B is formed around the second sensor housing 2061B. The second sensor insertion port 2058B (seal ring) of the chamber 2052 comes into contact with the second sealing surface 2068B, whereby the second internal space 2054B at the side of the second gas detection unit 2062B is enclosed.

The second sensor-side computer 2064B converts the analog response of the second gas detection unit 2062B into a digital signal and outputs the digital signal. The second sensor-side computer 2064B further normalizes the digital signal of the second gas detection unit 2062B and outputs the normalized digital signal. The second sensor-side computer 2064B also outputs its own second type information and second setting information stored in the second sensor-side memory. The second sensor-side computer 2064B receives command signals transmitted from the main body-side computer 34 or the base-side computer 2074, and outputs response signals based on the commands.

The second sensor-side connector 2065B is detachably connected to a second sensor receptacle connector 2075B of the sensor base 2070. The second gas sensor 2060B is fixed to the sensor base 2070 by the second sensor-side connector 2065B.

### (Sensor Base)

As shown in FIG. 16B, the sensor base 2070 includes a base housing 2071, a base-side circuit substrate 2073, the base-side computer 2074 and a base-side memory (not shown) mounted on the base-side circuit substrate 2073, the first sensor receptacle connector 2075A and the second sensor receptacle connector 2075B mounted on the base-side circuit substrate 2073, and a main body receptacle connector 2077 mounted on the base-side circuit substrate 2073.

As shown in FIGS. 17A and 17B, the base housing 2071 is a case of rectangular solid shape, and accommodates the base-side circuit substrate 2073 inside. A circular first sensor insertion opening 2071A is formed in the base housing 2071 at a position corresponding to the first sensor receptacle connector 2075A. As a result, the entire first sensor receptacle connector 2075A and a part of the base-side substrate 2073 are exposed outside. A circular second sensor insertion opening 2071B is formed in the base housing 2071 at a position corresponding to the second sensor receptacle connector 2075B. As a result, the entire second sensor receptacle connector 2075B and a part of the base-side substrate 2073 are exposed outside.

The first sensor receptacle connector 2075A and the first sensor-side connector 2065A are connected by inserting the first gas sensor 2060A through the first sensor insertion opening 2071A. Similarly, the second sensor receptacle connector 2075B and the second sensor-side connector 2065B are connected by inserting the second gas sensor 2060B through the second sensor insertion opening 2071B.

The base-side computer 2074 multiplexes the normalized digital signals received from both the first and second gas sensors 2060A and 2060B and transmits the resulting sequential signal to the main body-side computer 34. If at least either one of the first and second gas sensors 2060A and 2060B does not include a sensor-side computer, the base-side computer 2074 may digitize and normalize the analog signals of the gas detection units. The base-side computer 2074 can further output its own type information and various types of setting information stored in the base-side memory to the main body-side computer 34.

### (Sensor-Side Interface for Main Body)

As shown in FIG. 16C, the layout of a sensor-side interface for connecting the gas detection module 2050 and the main body 10 is defined by three locations that are the main body receptacle connector 2077, the chamber-side inflow channel 2052IN, and the chamber-side outflow channel 2052OUT. In other words, the sensor-side interface perfectly matches the main body-side interface of FIG. 4 (the layout of the main body-side connector 26A, the main body-side inlet 22IN, and the main body-side outlet 24OUT) and the sensor-side interface of the gas detection module 50 according to the first embodiment (FIG. 9C).

### <Connection Between Main Body and Gas Detection Module>

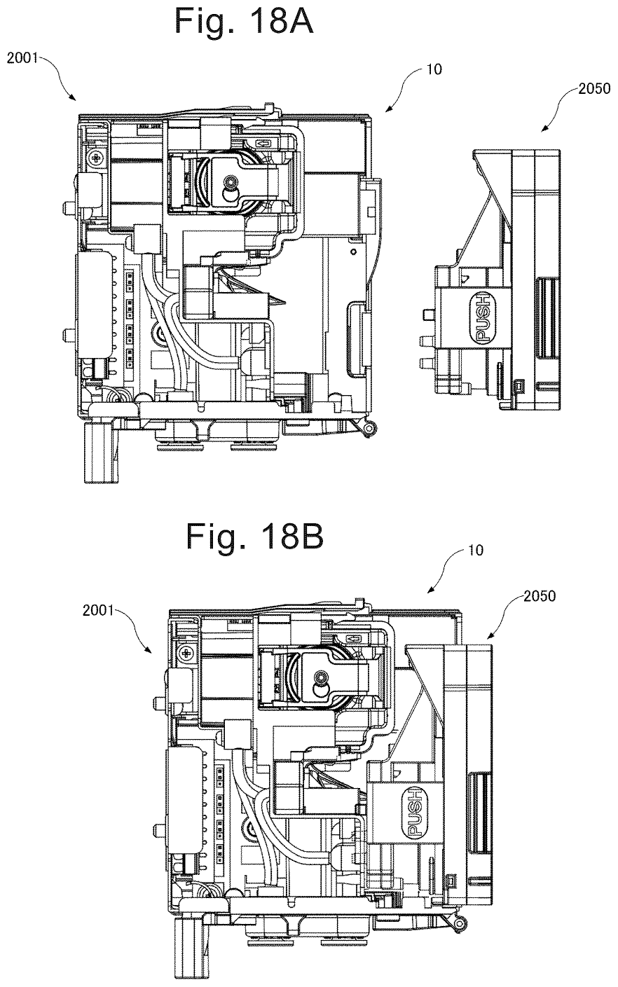

FIGS. 18A and 18B show a state of connection between the main body 10 and the gas detection module 2050 in the gas detector 2001. Since the main body-side interface (the layout of the main body-side connector 26A, the main body-side inlet 22IN, and the main body-side outlet 24OUT) and the sensor-side interface (the layout of the main body receptacle connector 2077, the chamber-side inflow channel 2052IN, and the chamber-side outflow channel 2052OUT) match perfectly, the main body 10 and the gas detection module 2050 are connected to each other by simply pushing the gas detection module 2050 into the main body 10 to the far side in the depth direction (see FIG. 18B). Conversely, the main body 10 and the gas detection module 2050 are separated from each other by pulling the gas detection module 2050 out of the main body 10 to the near side in the depth direction (see FIG. 18A).

### <Circuit Configuration>

FIG. 19 shows an overall circuit configuration of the gas detector 2001.

### (Base-Side Circuit Configuration)

The base-side circuit substrate 2073 includes the base-side computer 2074, the first sensor receptacle connector 2075A, the second sensor receptacle connector 2075B, the main body receptacle connector 2077, a first determination response unit 2702, a second determination response unit 2704, and a third determination power supply unit 2706.

The base-side circuit substrate 2073 includes a first base power path 106, a second base power path 108, and a heater power path 110 as paths for electrically connecting the main body receptacle connector 2077 and the first and second sensor receptacle connectors 2075A and 2075B. The first base power path 106 extending from the main body receptacle connector 2077 branches midway into a first sensor first base power path 106A and a second sensor first base power path 106B, which are connected to the first sensor receptacle connector 2075A and the second sensor receptacle connector 2075B, respectively. The second base power path 108 extending from the main body receptacle connector 2077 branches midway into a first sensor second base power path 108A and a second sensor second base power path 108B, which are connected to the first sensor receptacle connector 2075A and the second sensor receptacle connector 2075B, respectively. The heater power path 110 extending from the main body receptacle connector 2077 branches midway into a first sensor heater power path 110A and a second sensor heater power path 110B, which are connected to the first sensor receptacle connector 2075A and the second sensor receptacle connector 2075B, respectively.

The base-side circuit substrate 2073 further includes a first determination path 102, a second determination path 104, and a third determination path 116 as electrical paths connected to the main body receptacle connector 2077. The first determination response unit 2702 is connected to the first determination path 102. The second determination response unit 2704 is connected to the second determination path 104. The third determination power supply unit 2706 is connected to the third determination path 116.

The base-side circuit substrate 2073 includes a first communication path 112, a reset path 114, and a second communication path 118 as signal transmission paths extending from the main body receptacle connector 2077 to the base-side computer 2074.

The base-side circuit substrate 2073 includes a one-side first communication path 112A, a first reset path 114A, a one-side second communication path 118A, a first sensor-side first determination path 120A, and a first sensor-side second determination path 121A as communication paths extending from the base-side computer 2074 to the first sensor receptacle connector 2075A. The base-side circuit substrate 2073 includes an other-side first communication path 112B, a second reset path 114B, an other-side second communication path 118B, a second sensor-side first determination path 120B, and a second sensor-side second determination path 121B as communication paths extending from the base-side computer 2074 to the second sensor receptacle connector 2075B.

As a result, the second communication path 118 extending from the main body receptacle connector 2077 branches at the base-side computer 2074 into the one-side second communication path 118A and the other-side second communication path 118B, which extend to the first sensor receptacle connector 2075A and the second sensor receptacle connector 2075B. The multiplexed sequential commands that the base-side computer 2074 receives from the main body-side computer 34 are thereby separated at the base-side computer 2074 and transmitted to the one-side second communication path 118A and the other-side second communication path 118B in a distributed manner.

Meanwhile, the one-side first communication path 112A extending from the first sensor receptacle connector 2075A and the other-side first communication path 112B extending from the second sensor receptacle connector 2075B merge at the base-side computer 2074 into the first communication path 112, which extends to the main body receptacle connector 2077. The normalized output signals (measurement results) received from the first gas sensor 2060A and the second gas sensor 2060B are thus multiplexed by the base-side computer 2074 and transmitted to the first communication path 112.

The reset path 114 extending from the main body receptacle connector 2077 branches at the base-side computer 2074 into the first reset path 114A and the second reset path 114B, which extend to the first sensor receptacle connector 2075A and the second sensor receptacle connector 2075B. The reset signal transmitted from the main body-side computer 34 is thereby distributed and transmitted to the first reset path 114A and the second reset path 114B.

### (First Sensor-Side Circuit Configuration)

The first sensor-side circuit substrate 2063A includes the first sensor-side computer 2064A, the first sensor-side connector 2065A, the first sensor-side first determination response unit 2707A, and the first sensor-side second determination response unit 2708A. The first gas detection unit 2062A is further connected to the first sensor-side circuit substrate 2063A. A first heater unit 2062A H such as a noble metal coil can be built in the first gas detection unit 2062A.

The first sensor-side circuit substrate 2063A includes a first sensor first base power path 106A, a first sensor second base power path 108A, a first sensor heater power path 110A, a one-side first communication path 112A, a first reset path 114A, a one-side second communication path 118A, a first sensor-side first determination path 120A, and a first sensor-side second determination path 121A as electrical paths connected to the sensor base 2070 via the first sensor-side connector 2065A.

The base-side computer 2074 supplies first sensor-side first determination power to the first sensor-side first determination response unit 2707A of the first gas sensor 2060A via the first sensor-side first determination path 120A. The base-side computer 2074 further supplies first sensor-side second determination power to the first sensor-side second determination response unit 2708A of the first gas sensor 2060A via the first sensor-side second determination path 121A.

The base-side computer 2074 includes a first sensor-side first determination unit (not shown) that detects the voltage level of the first sensor-side first determination path 120A, and a first sensor-side second determination unit (not shown) that detects the voltage level of the first sensor-side second determination path 121A.

The first sensor-side first determination response unit 2707A is a first sensor first response-side resistor connected between the first sensor-side first determination path 120A and the ground, for example. The first sensor-side first determination response unit 2707A sets the voltage level of the first sensor-side first determination path 120A. For example, when the base-side computer 2074 supplies a voltage of 3.3 V, the voltage level of the first sensor-side first determination path 120A is the divided voltage between a first base-side resistor in the base-side computer 2074 and the first sensor first response-side resistor. In other words, if the value of the first base-side resistor is fixed, the voltage level of the first sensor-side first determination path 120A can be uniquely determined by using the value of the first sensor first response-side resistor. This voltage level is detected by the first sensor-side first determination unit of the base-side computer 2074. Whether the first gas sensor 2060A is connected and the type of the first gas sensor 2060A (including the type of dummy gas sensor mounted for test purposes) can thus be determined by appropriately setting the value of the first sensor first response-side resistor of the first sensor-side first determination response unit 2707A.

The result of determination of the type and presence of the first gas sensor 2060A by the base-side computer 2074 is transmitted to the main body-side computer 34 as a digital signal via the first communication path 112.

The first sensor-side second determination response unit 2708A is a first sensor second response-side resistor connected between the first sensor-side second determination path 121A and the ground, for example. The first sensor-side second determination response unit 2708A sets the voltage level of the first sensor-side second determination path 121A. For example, when the base-side computer 2074 supplies a voltage of 3.3 V, the voltage level of the first sensor-side second determination path 121A is the divided voltage between the first base-side resistor in the base-side computer 2074 and the first sensor second response-side resistor. In other words, if the value of the first base-side resistor is fixed, the voltage level of the first sensor-side second determination path 121A can be uniquely determined by using the value of the first sensor second response-side resistor. This voltage level is detected by the first sensor-side second determination unit of the base-side computer 2074. Whether the first gas sensor 2060A is connected and the type of the first gas sensor 2060A (including the type of dummy gas sensor mounted for test purposes) can thus be determined by appropriately setting the value of the first sensor second response-side resistor of the first sensor-side second determination response unit 2708A.

The result of determination of the type and presence of the first gas sensor 2060A by the base-side computer 2074 is transmitted to the main body-side computer 34 as a digital signal via the first communication path 112.

The analog output of the first gas detection unit 2062A is transmitted to the first sensor-side computer 2064A via a first analog output path 2130A. The first sensor-side computer 2064A converts the received analog signal into a digital signal, normalizes the digital signal, and then outputs the normalized digital signal through the one-side first communication path 112A.

The first sensor first base power path 106A and the first sensor second base power path 108A extending from the first sensor-side connector 2065A are connected to the first gas detection unit 2062A. The first base power and/or the second base power are thereby supplied to the first gas detection unit 2062A. The first sensor heater power path 110A extending from the first sensor-side connector 2065A is connected to the first heater unit 2062A H. The heater power is thereby supplied to the first heater unit 2062A H.

### (Second Sensor-Side Circuit Configuration)

The second sensor-side circuit substrate 2063B includes the second sensor-side computer 2064B, the second sensor-side connector 2065B, the second sensor-side first determination response unit 2707B, and the second sensor-side second determination response unit 2708B. The second gas detection unit 2062B is further connected to the second sensor-side circuit substrate 2063B. A second heater unit 2062B H such as a noble metal coil can be built in the second gas detection unit 2062B.

The second sensor-side circuit substrate 2063B includes a second sensor first base power path 106B, a second sensor second base power path 108B, a second sensor heater power path 110B, an other-side first communication path 112B, a second reset path 114B, an other-side second communication path 118B, a second sensor-side first determination path 120B, and a second sensor-side second determination path 121B as electrical paths connected to the sensor base 2070 via the second sensor-side connector 2065B.

The base-side computer 2074 supplies second sensor-side first determination power to the second sensor-side first determination response unit 2707B of the second gas sensor 2060B via the second sensor-side first determination path 120B. The base-side computer 2074 further supplies second sensor-side second determination power to the second sensor-side second determination response unit 2708B of the second gas sensor 2060B via the second sensor-side second determination path 121B.

The base-side computer 2074 includes a second sensor-side first determination unit (not shown) that detects the voltage level of the second sensor-side first determination path 120B, and a second sensor-side second determination unit (not shown) that detects the voltage level of the second sensor-side second determination path 121B.

The second sensor-side first determination response unit 2707B is a second sensor first response-side resistor connected between the second sensor-side first determination path 120B and the ground, for example. The second sensor-side first determination response unit 2707B sets the voltage level of the second sensor-side first determination path 120B. For example, when the base-side computer 2074 supplies a voltage of 3.3 V, the voltage level of the second sensor-side first determination path 120B is the divided voltage between a second base-side resistor in the base-side computer 2074 and the second sensor first response-side resistor. In other words, if the value of the second base-side resistor is fixed, the voltage level of the second sensor-side first determination path 120B can be uniquely determined by using the value of the second sensor first response-side resistor. This voltage level is detected by the second sensor-side first determination unit of the base-side computer 2074. Whether the second gas sensor 2060B is connected and the type of the second gas sensor 2060B (including the type of dummy gas sensor mounted for test purposes) can thus be determined by appropriately setting the value of the second sensor first response-side resistor of the second sensor-side first determination response unit 2707B.

The result of determination of the type and presence of the second gas sensor 2060B by the base-side computer 2074 is transmitted to the main body-side computer 34 as a digital signal via the first communication path 112.

The second sensor-side second determination response unit 2708B is a second sensor second response-side resistor connected between the second sensor-side second determination path 121B and the ground, for example. The second sensor-side second determination response unit 2708B sets the voltage level of the second sensor-side second determination path 121B. For example, when the base-side computer 2074 supplies a voltage of 3.3 V, the voltage level of the second sensor-side second determination path 121B is the divided voltage between the second base-side resistor in the base-side computer 2074 and the second sensor second response-side resistor. In other words, if the value of the second base-side resistor is fixed, the voltage level of the second sensor-side second determination path 121B can be uniquely determined by using the value of the second sensor second response-side resistor. This voltage level is detected by the second sensor-side second determination unit of the base-side computer 2074. Whether the second gas sensor 2060B is connected and the type of the second gas sensor 2060B (including the type of dummy gas sensor mounted for test purposes) can thus be determined by appropriately setting the value of the second sensor second response-side resistor of the second sensor-side second determination response unit 2708B.

The result of determination of the type and presence of the second gas sensor 2060B by the base-side computer 2074 is transmitted to the main body-side computer 34 as a digital signal via the first communication path 112.

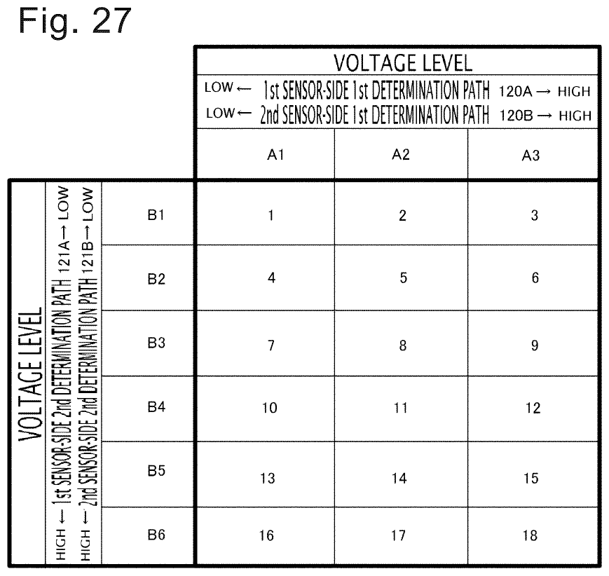

In the present embodiment, the base-side computer 2074 desirably determines the gas sensors by referring to a sensor determination table shown in FIG. 27, stored in its own memory. This sensor determination table determines the voltage level of the first sensor-side first determination path 120A or the second sensor-side first determination path 120B in multiple grades (here, three grades A1 to A3) and the voltage level of the first sensor-side second determination path 121A or the second sensor-side second determination path 121B in multiple grades (six grates B1 to B6). As a result, the base-side computer 2074 can determine a total of 18 types of gas sensors on the basis of the combinations of the grades.

Duplicating the determination of the type of the sensor base 2070 by the main body-side computer 34 using the first and second determination response units 2702 and 2704 of the base-side circuit substrate 2073 and the determination of the types of the first and second gas sensors 2060A and 2060B by the base-side computer 2074 using the first sensor-side first determination response unit 2707A, the first sensor-side second determination response unit 2708A, the second sensor-side first determination response unit 2707B, and the second sensor-side second determination response unit 2708B as described above enables individual detection of omission, a failure, and the like of the first gas sensor 2060A and/or the second gas sensor 2060B. The determination result of the base-side computer 2074 is converted into a digital signal by the base-side computer 2074 and transmitted via the first communication path 112. This can prevent an increase in the number of pins of the main body receptacle connector 2077.

The analog output of the second gas detection unit 2062B is transmitted to the second sensor-side computer 2064B via a second analog output path 2130B. The second sensor-side computer 2064B converts the received analog signal into a digital signal, normalizes the digital signal, and then outputs the normalized digital signal through the other-side first communication path 112B.

The second sensor first base power path 106B and the second sensor second base power path 108B extending from the second sensor-side connector 2065B are connected to the second gas detection unit 2062B. The first base power and/or the second base power are thereby supplied to the second gas detection unit 2062B. The second sensor heater power path 110B extending from the second sensor-side connector 2065B is connected to the second heater unit 2062B H. The heater power is thereby supplied to the second heater unit 2062B H.

### (Numbers of Pins of Connectors)

In the present embodiment, the number of pins (number of paths) of the first sensor-side connector 2065A is eight, the number of pins of the second sensor-side connector 2065B is eight, and the total number of pins is thus 16, excluding the ground pins GND. The number of pins (number of paths) of the main body-side connector 26A is eight, excluding the ground pins GND. In other words, the total number of pins of the main body-side connector 26A connecting the sensor base 2070 and the main body 10 is set to be smaller than the total number of pins of the first and second gas sensors 2060A and 2060B. As a result, the main body-side connector 26A can be made compact. The reduction in the number of pins of the main body-side connector 26A is implemented by the multiplexer/demultiplexer processing of commands and output signals and the single-path function switching processing.

### <Gas Detection Program>

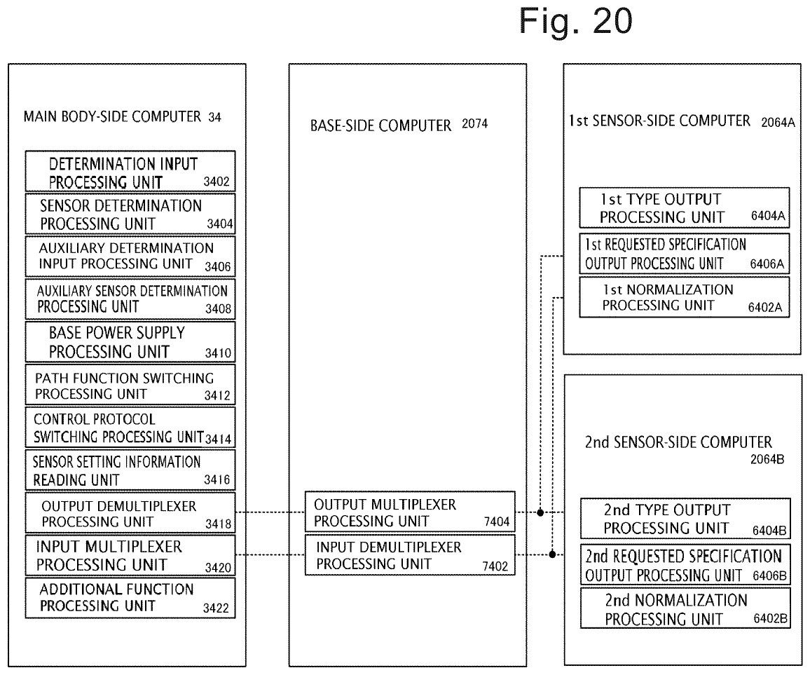

FIG. 20 shows functions that the main body-side computer 34, the base-side computer 2074, the first sensor-side computer 2064A, and the second sensor-side computer 2064B of the gas detector 2001 implement by executing a gas detection program (gas detection procedure). The functional configurations of the main body-side computer 34 and the base-side computer 2074 are the same as those according to the first embodiment. A description thereof will thus be omitted.

### (Functions of First Sensor-Side Computer)

The first sensor-side computer 2064A includes a first normalization processing unit 6402A, a first type output processing unit 6404A, and a first requested specification output processing unit 6406A. The first normalization processing unit 6402A digitizes the analog output obtained from the first gas sensor 2060A, and normalizes the resulting digital output according to an output rule (output specification) set by the main body-side computer 34 in advance.

The first type output processing unit 6404A outputs first type information (for example, information about the gas detection method of the first gas sensor) stored in the sensor-side memory (not shown) mounted on the first sensor-side circuit substrate 2063A to the main body-side computer 34 as a response based on the auxiliary command transmitted from the auxiliary determination input processing unit 3406 of the main body-side computer 34.

The first requested specification output processing unit 6406A outputs first requested specification information (for example, information about the base voltage level of the first gas sensor, whether the heater power is needed, and the level of the heater power) stored in the sensor-side memory (not shown) mounted on the first sensor-side circuit substrate 2063A to the main body-side computer 34 as a response to the command transmitted from the sensor setting information reading unit 3416 of the main body-side computer 34. The sensor setting information reading unit 3416 of the main body-side computer 34 can thereby obtain the setting information about the first gas sensor 2060A.

### (Functions of Second Sensor-Side Computer)

The second sensor-side computer 2064B includes a second normalization processing unit 6402B, a second type output processing unit 6404B, and a second requested specification output processing unit 6406B. The second normalization processing unit 6402B digitizes the analog output obtained from the second gas sensor 2060B, and normalizes the resulting digital output according to an output rule (output specification) set by the main body-side computer 34 in advance.

The second type output processing unit 6404B outputs second type information (for example, information about the gas detection method of the second gas sensor) stored in the sensor-side memory (not shown) mounted on the second sensor-side circuit substrate 2063B to the main body-side computer 34 as a response based on the auxiliary command transmitted from the auxiliary determination input processing unit 3406 of the main body-side computer 34.

The second requested specification output processing unit 6406B outputs second requested specification information (for example, information about the base voltage level of the second gas sensor, whether the heater power is needed, and the level of the heater power) stored in the sensor-side memory (not shown) mounted on the second sensor-side circuit substrate 2063B to the main body-side computer 34 as a response to the command transmitted from the sensor setting information reading unit 3416 of the main body-side computer 34. The sensor setting information reading unit 3416 of the main body-side computer 34 can thereby obtain the setting information about the second gas sensor 2060B.

### (Functions of Base-Side Computer)

The input demultiplexer processing unit 7402 of the base-side computer 2074 distributes the multiplexed sequential commands transmitted from the input multiplexer processing unit 3420 of the main body-side computer 34 to the first sensor-side computer 2064A and the second sensor-side computer 2064B. The output multiplexer processing unit 7404 of the base-side computer 2074 multiplexes the normalized measurement result received from the first sensor-side computer 2064A and the normalized measurement result received from the second sensor-side computer 2064B into a sequential output, and transmits the sequential output to the main body-side computer 34. With such a configuration, the main body-side computer 34 can simultaneously control the plurality of gas sensors 2060A and 2060B built in the gas detection module 2050.

(Procedure from Main Power Supply ON to Start of Measurement)

The procedure from the main power supply ON to the start of measurement according to the second embodiment is the same as that described with reference to the flowchart of FIG. 15 according to the first embodiment. A description thereof will thus be omitted here.

### Third Embodiment

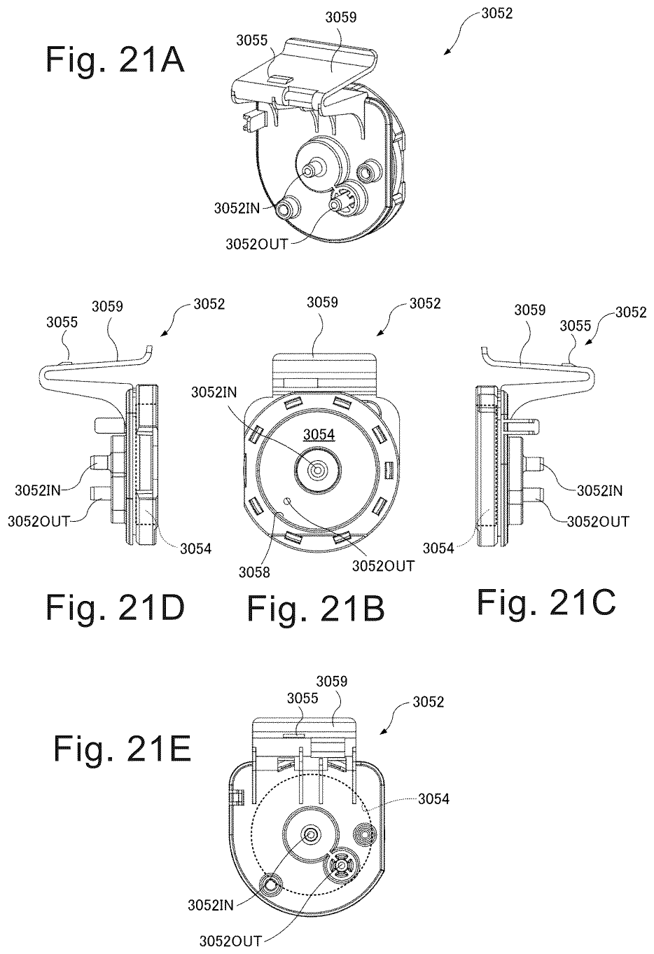

A gas detector 3001 according to a third embodiment will be described with reference to FIGS. 21A to 21E and subsequent figures. The gas detector 3001 includes the same main body 10 as that of the first embodiment. A description thereof will thus be omitted. Differences of a gas detection module 3050 detachably connected to the main body 10 from the gas detection module 50 according to the first embodiment will mainly be described. The same or similar parts and members between the gas detection module 3050 and the gas detection module 50 are denoted by reference numerals with the same lower three digits, and a detailed description thereof will be omitted.

### <Gas Detection Module>

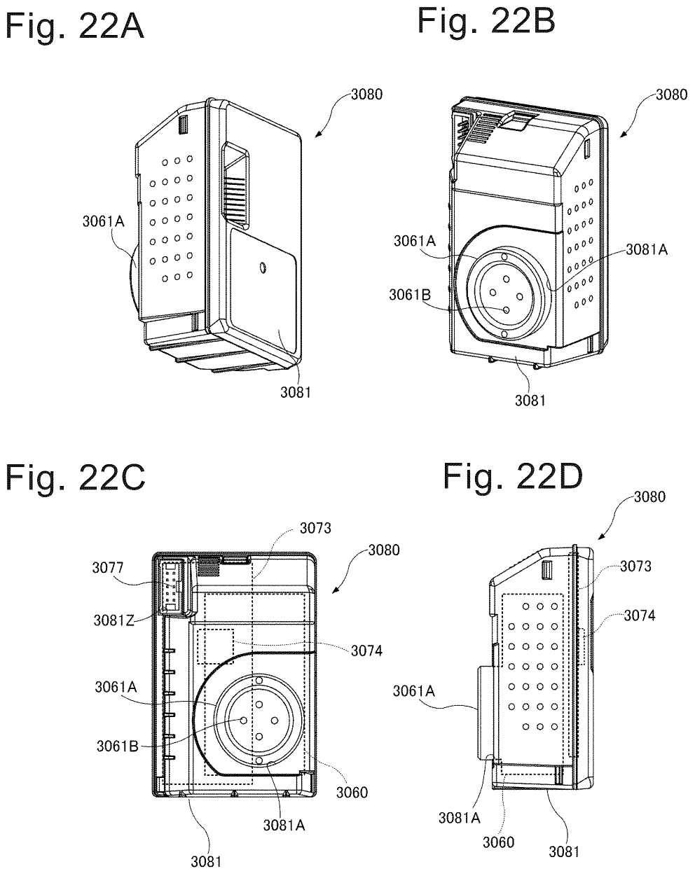

The gas detection module 3050 includes a chamber 3052 shown in FIGS. 21A to 21E and a sensor unit 3080 shown in FIGS. 22A to 22D. The chamber 3052 and the sensor unit 3080 can be separated from each other.

### (Chamber)

As shown in FIGS. 21A to 21E, the chamber 3052 includes a chamber-side inflow channel 3052IN, a chamber-side outflow channel 3052OUT, and an internal space 3054. The chamber-side inflow channel 3052IN of male structure (protruding structure) is detachably connected to the main body-side inlet 22IN. The chamber-side outflow channel 3052OUT of male structure (protruding structure) is detachably connected to the main body-side outlet 24OUT. The internal space 3054 is interposed between the chamber-side inflow channel 3052IN and the chamber-side outflow channel 3052OUT. The internal space 3054 is a cylindrical (circular disk-like) space with a bottom, and the annular rim of the cylinder serves as a unit insertion port 3058. A coupling projection 3061A (to be described below) of the sensor unit 3080 is inserted into the unit insertion port 3058. The chamber-side inflow channel 3052IN and the chamber-side outflow channel 3052OUT communicate with the bottom of the internal space 3054. A rubber seal ring is attached to the unit insertion port 3058. This unit insertion port 3058 (seal ring) is brought into contact with the peripheral rim of the coupling projection 3061A (to be described below) of the sensor unit 3080, whereby the internal space 3054 is enclosed.

The chamber 3052 includes an elastic portion 3059 protruding in a U shape toward the far side in the depth direction when the chamber 3052 is oriented for attachment to the main body 10. The elastic portion 3059 is elastically displaceable in the vertical direction. The elastic portion 3059 includes a chamber-side engagement portion 3055 having a claw structure.

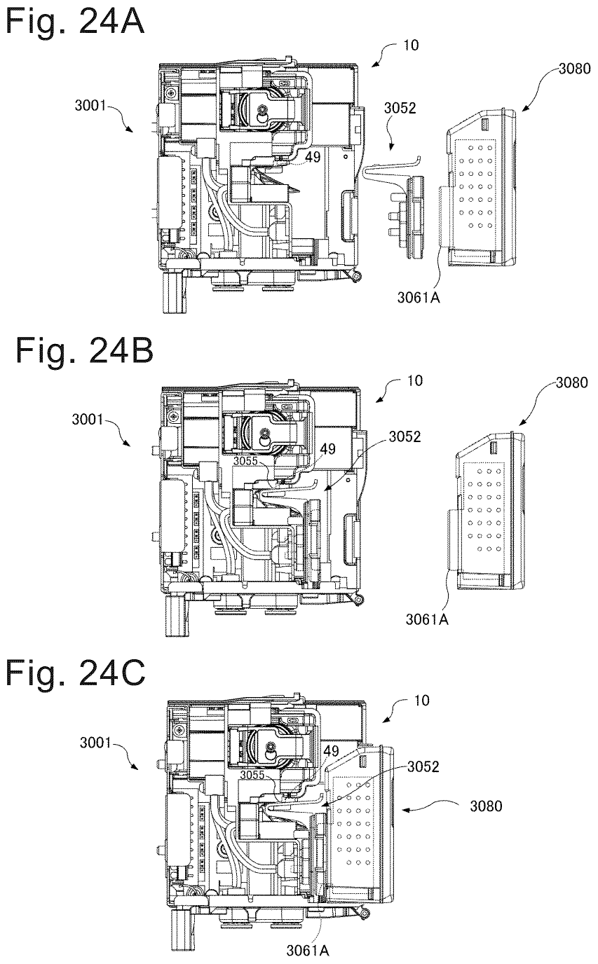

The chamber 3052 is fixed to the main body 10 by connecting the chamber-side inflow channel 3052IN and the chamber-side outflow channel 3052OUT to the main body-side inlet 22IN and the main body-side outlet 24OUT, and engaging the chamber-side engagement portion 3055 with a main body-side engagement portion 49 (see FIGS. 24A to 24C) that is a receptacle groove structure of the main body 10.

### (Sensor Unit)

As shown in FIGS. 22A to 22D, the sensor unit 3080 includes a unit housing 3081, a gas sensor 3060, a main body receptacle connector 3077 disposed on the unit housing 3081, a unit-side circuit substrate 3073, a unit-side computer 3074, and a unit-side memory (not shown).

The unit housing 3081 is a case of rectangular solid shape, and accommodates the gas sensor 3060, the unit-side circuit substrate 3073, and the unit-side computer 3074 inside. The gas sensor 3060 is electrically directly connected to the unit-side circuit substrate 3073 inside the unit housing 3081. As a result, the sensor unit 3080 is configured so that the gas sensor 3060 is not replaceable by itself in a freely attachable and detachable manner.

The gas sensor 3060 has the coupling projection 3061A that projects cylindrically. The end face of this coupling projection 3061A serves as the detection surface of the gas sensor 3060. A plurality (here, four) of air passages 3061B for taking gas into the gas sensor 3060 are formed in the end face. The unit housing 3081 has an opening 3081A for exposing and protruding the coupling projection 3061A of the gas sensor 3060 to the outside. The coupling projection 3061A of the gas sensor 3060 protruded from the opening 3081A is inserted into the unit insertion port 3058 of the chamber 3052, whereby the internal space 3054 of the chamber 3052 is enclosed.

In the present embodiment, the coupling projection 3061A that constitutes a part of the housing of the gas sensor 3060 and the unit housing 3081 are described to be separate members. However, the present invention is not limited thereto. The unit housing 3081 itself may be configured to constitute a part of the housing of the gas sensor 3060. To put it another way, the coupling projection 3061A of the gas sensor 3060 constitutes a part of the unit housing 3081 when the sensor unit 3080 is viewed as a whole.

The unit-side computer 3074 is mounted on the unit-side circuit substrate 3073. The unit-side computer 3074 converts an analog response output from the gas sensor 3060 into a digital signal and outputs the digital signal. The unit-side computer 3074 further normalizes the digital signal. The unit-side computer 3074 also outputs its own type information and setting information stored in the unit-side memory. The unit-side computer 3074 receives command signals transmitted from the main body-side computer 34 and outputs response signals based on the commands.

Although not shown in particular, if a plurality of gas sensors are built in the unit housing 3081, the unit-side computer 3074 may multiplex the normalized digital signals from the respective gas sensors and transmit the resulting sequential signal to the main body-side computer 34.

A rectangular main body connection opening 3081Z is formed in the unit housing 3081 at a position corresponding to the main body receptacle connector 3077 of the unit-side circuit substrate 3073, whereby the main body receptacle connector 3077 is exposed.

### (Sensor-Side Interface for Main Body)

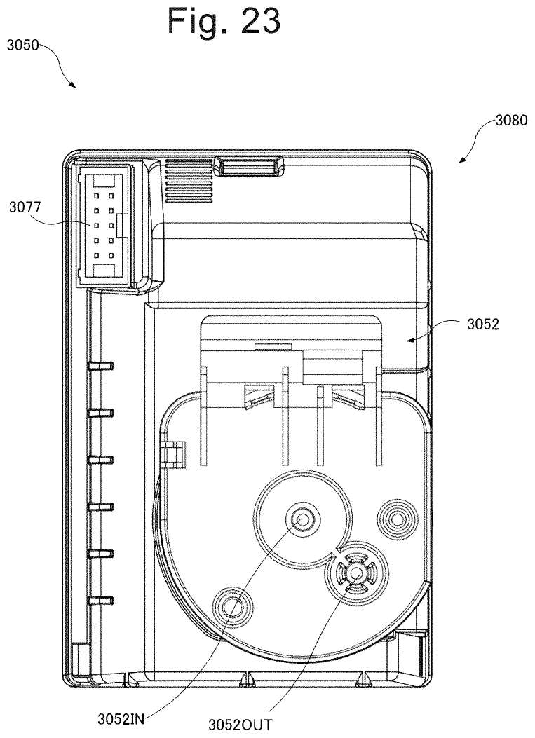

FIG. 23 shows the gas detection module 3050 constituted by coupling the sensor unit 3080 and the chamber 3052. The layout of the sensor-side interface for connecting the gas detection module 3050 and the main body 10 is defined by three locations that are the main body receptacle connector 3077, the chamber-side inflow channel 3052IN, and the chamber-side outflow channel 3052OUT. In other words, this sensor-side interface perfectly matches the main body-side interface of FIG. 4 (the layout of the main body-side connector 26A, the main body-side inlet 22IN, and the main body-side outlet 24OUT), the sensor-side interface of the gas detection module 50 according to the first embodiment, and the sensor-side interface of the gas detection module 2050 according to the second embodiment.

### <Connection Between Main Body and Gas Detection Module>

FIGS. 24A to 24C show a state of connection between the main body 10 and the gas detection module 3050 in the gas detector 3001. The main body-side interface (the layout of the main body-side connector 26A, the main body-side inlet 22IN, and the main body-side outlet 24OUT) and the sensor-side interface (the layout of the main body receptacle connector 3077, the chamber-side inflow channel 3052IN, and the chamber-side outflow channel 3052OUT) match perfectly. As shown in FIG. 24B, the chamber 3052 is initially connected to the main body 10. Here, the chamber 3052 is fixed to the main body 10 by engaging the chamber-side engagement portion 3055 with the main body-side engagement portion 49 of the main body 10.

As shown in FIG. 24C, the sensor unit 3080 is then attached by pushing the sensor unit 3080 into the main body 10 and the chamber 3052, which are integrated with each other, to the far side in the depth direction. Conversely, the sensor unit 3080 is separated by simply pulling the sensor unit 3080 out of the main body 10 and the chamber 3052 to the near side in the depth direction (see FIG. 24B). In replacing the sensor unit 3080 with another sensor unit 3080 of a different gas detection method, only the sensor units 3080 can thus be easily detached and attached with the chamber 3052 left in the main body 10. If the gas detection module 3050 is replaced with the gas detection module 50 according to the first embodiment or the gas detection module 2050 according to the second embodiment, the chamber 3052 can be detached from the main body 10 by deforming the elastic portion 3059 to disengage the chamber-side engagement portion 3055 from the main body-side engagement portion 49.

### <Circuit Configuration>

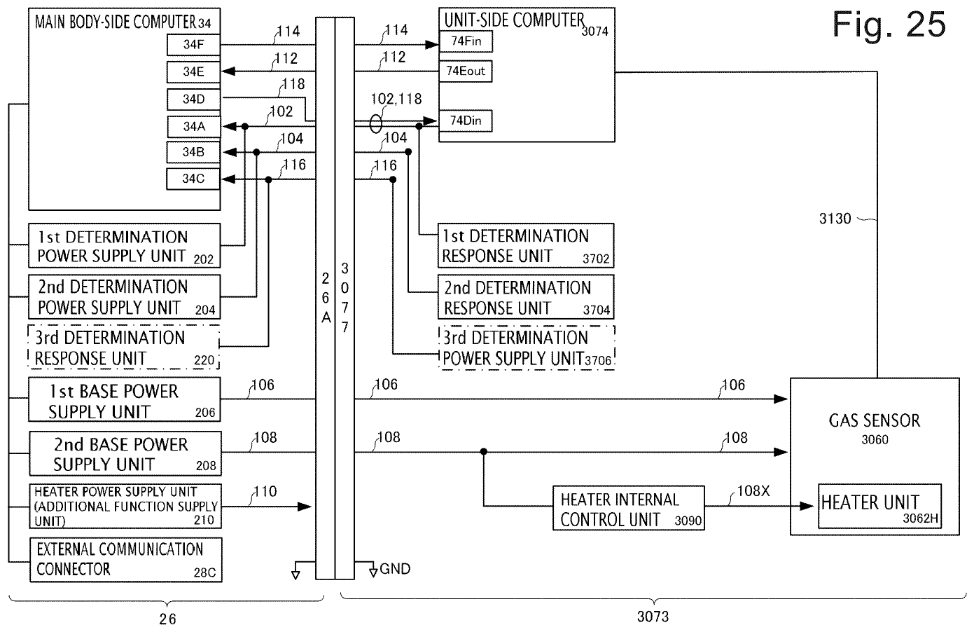

FIG. 25 shows an overall circuit configuration of the gas detector 3001.

### (Unit-Side Circuit Configuration)

The unit-side circuit substrate 3073 includes the unit-side computer 3074, the main body receptacle connector 3077, a first determination response unit 3702, a second determination response unit 3704, and a third determination power supply unit 3706.

The unit-side circuit substrate 3073 includes a first base power path 106 and a second base power path 108 as paths for electrically connecting the main body receptacle connector 3077 and the gas sensor 3060. The first base power and/or the second base power are thereby supplied to the gas sensor 3060. The unit-side circuit substrate 3073 includes a heater power path 108X using the second base power path 108. The heater power is controlled by a heater internal control unit 3090. The heater power is supplied to a heater unit 3062H such as a noble metal coil. The power supply from the heater power supply unit 210 on the main body-side circuit substrate 26 is therefore stopped.

The unit-side circuit substrate 3073 further includes a first determination path 102, a second determination path 104, and a third determination path 116 as electrical paths connected to the main body receptacle connector 3077. The first determination response unit 3702 is connected to the first determination path 102. The second determination response unit 3704 is connected to the second determination path 104. The third determination power supply unit 3706 is connected to the third determination path 116.

The unit-side circuit substrate 3073 includes a first communication path 112, a reset path 114, and a second communication path 118 as signal transmission paths extending from the main body receptacle connector 3077 to the unit-side computer 3074.

An analog signal output from the gas sensor 3060 is transmitted to the unit-side computer 3074 via an analog output path 3130. The unit-side computer 3074 converts the received analog signal into a digital signal, normalizes the digital signal, and then outputs the normalized digital signal through the first communication path 112.

### <Gas Detection Program>

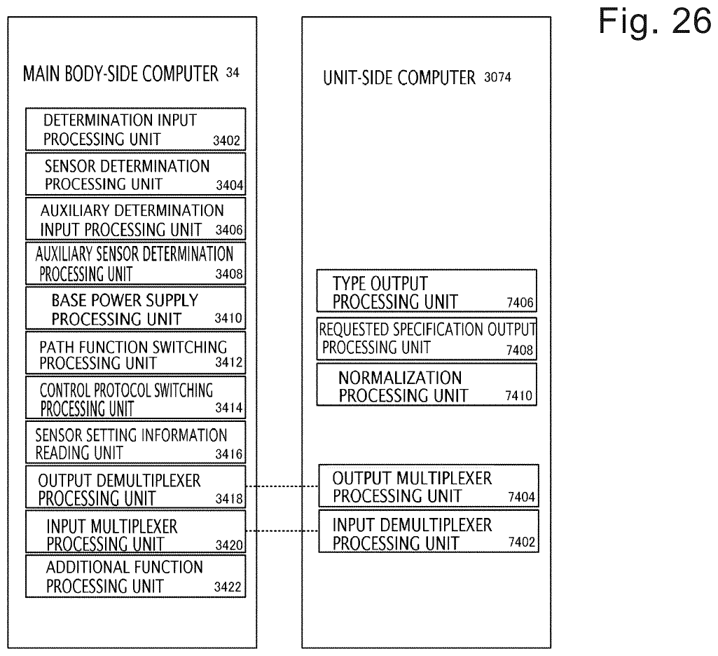

FIG. 26 shows functions that the main body-side computer 34 and the unit-side computer 3074 of the gas detector 3001 implement by performing a gas detection program (gas detection procedure). Since the functional configuration of the main body-side computer 34 is the same as that in the first and second embodiments, a description thereof will be omitted.

### (Functions of Unit-Side Computer)

The unit-side computer 3074 includes a normalization processing unit 7410, a type output processing unit 7406, and a requested specification output processing unit 7408. The normalization processing unit 7410 digitizes the analog output obtained from the gas sensor 3060, and normalizes the resulting digital output according to an output rule (output specification) set by the main body-side computer 34 in advance.

The type output processing unit 7406 outputs type information (such as information about the gas detection method of the gas sensor) stored in the unit-side memory (not shown) mounted on the unit-side circuit substrate 3073 to the main body-side computer 34 as a response based on the auxiliary command transmitted from the auxiliary determination input processing unit 3406 of the main body-side computer 34.

The requested specification output processing unit 7408 outputs requested specification information (for example, information about the base voltage level of the gas sensor and information indicating that heater power is not needed) stored in the sensor-side memory (not shown) mounted on the unit-side circuit substrate 3073 to the main body-side computer 34 as a response to the command transmitted from the sensor setting information reading unit 3416 of the main body-side computer 34. The sensor setting information reading unit 3416 of the main body-side computer 34 can thereby obtain the setting information about the gas sensor 3060.

The unit-side computer 3074 further includes an input demultiplexer processing unit 7402 and an output multiplexer processing unit 7404. The input demultiplexer processing unit 7402 distributes the multiplexed sequential commands transmitted from the input multiplexer processing unit 3420 of the main body-side computer 34. The unit-side computer 3074 is suitably used when a plurality of gas sensors are built in the sensor unit 3080.

When a plurality of gas sensors are built in the sensor unit 3080, the output multiplexer processing unit 7404 multiplexes the normalized measurement results received from the plurality of gas sensors into a sequential output, and transmits the sequential output to the main body-side computer 34. With such a configuration, the main body-side computer 34 can simultaneously control the plurality of gas sensors mounted on the gas detection module 3050.

A comparison between the unit-side computer 3074 according to this third embodiment and the base-side and sensor-side computers 74 and 64 according to the first embodiment shows that the unit-side computer 3074 integrates the functions of both the base-side and sensor-side computers 74 and 64 according to the first embodiment. In other words, the unit-side computer 3074 is a concept including the base-side and sensor-side computers 74 and 64.

(Procedure from Main Power Supply ON to Start of Measurement)

The procedure from the main power supply ON to the start of measurement according to the third embodiment is the same as that according to the first embodiment described with reference to the flowchart of FIG. 15. A description thereof will thus be omitted.

As described above, the gas detectors 1 and 2001 according to the first and second embodiments include the common main body 10, the chamber 52 or 2052 separably (detachably) disposed on the main body 10, the gas sensor 60 or gas sensors 2060A and 2060B separably (detachably) disposed on the chamber, and the sensor base 70 or 2070 separably (detachably) disposed on the gas sensor(s) and the main body.

An optimum gas sensor or sensors can thus be freely selected and used from various types of gas sensors, depending on the use purposes. Moreover, if a gas sensor or sensor base fails, only the gas sensor or sensor base can be replaced. This can reduce the maintenance burden.

In the gas detectors 1 and 2001 according to the first and second embodiments, the chamber 52 or 2052 has the sensor insertion port 58 or sensor insertion ports 2058A and 2058B into which the gas sensor 60 or gas sensors 2060A and 2060B are inserted. The gas sensors 60, 2060A, and 2060B have an annular sensor-side surface 68, 2068A, or 2068B that can abut against the sensor insertion port. As a result, the sensor insertion port 58 or sensor insertion ports 2058A and 2058B can be brought into contact with the sensor-side sealing surface 68 or sensor-side sealing surfaces 2068A and 2068B by simply swinging and fixing the chamber 52 or 2052 to the sensor base 70 or 2070 to enclose the internal space 54 or internal spaces 2054A and 2054B.

Here, the chambers 52 and 2052 include the chamber-side engagement portions 55 or 2055, and the sensor bases 70 and 2070 include the base-side engagement portions 79 or 2079 to be engaged with the chamber-side engagement portions 55 or 2055. The chambers 52 and 2052 can thus be easily fixed to the sensor bases 70 and 2070. Moreover, the chambers 52 and 2052 can be moved (swung) relative to the sensor base 70 or 2070 by disengaging the engagement portions from each other. The gas sensors 60, 2060A, and 2060B can thus be easily replaced.

In particular, in the case of the gas detection module 2050 described with the gas detector 2001 according to the second embodiment, the plurality of gas sensors 2060A and 2060B can be mounted on the sensor base 2070. This enables simultaneous detection of a plurality of target gases, or detection of the same target gas using different techniques. The base-side computer 2074 can distribute the multiplexed sequential commands transmitted from the main body-side computer 34 to the plurality of gas sensors 2060A and 2060B using the input demultiplexer processing unit 7402. Moreover, the base-side computer 2074 can multiplex and convert the normalized measurement results received from the plurality of gas sensors 2060A and 2060B into a sequential output using the output multiplexer processing unit 7404, and output the multiplexed sequential output to the main body-side computer 34. With such a configuration, the main body-side computer 34 can simultaneously control the plurality of gas sensors 2060A and 2060B built in the gas detection module 2050.

As demonstrated by the gas detectors 1, 2001, and 3001 according to the first to third embodiments, the sensor-side interfaces of the gas detection module 50 according to the first embodiment, the gas detection module 2050 according to the second embodiment, and the gas detection module 3050 according to the third embodiment match perfectly. The three types of gas detection modules 50, 2050, and 3050 can thus be freely replaced on the common main body 10.

For example, there can be gas detection methods like those of the gas detection modules 50 and 2050 according to the first and second embodiments where the gas sensors 60, 2060A, and 2060B are small in size and can be handled by themselves, and gas detection methods like that of the gas detection module 3050 according to the third embodiment where the gas sensor 3060 is integrally built in the sensor unit 3080 and should not be directly handled by itself.

If the specifications of the gas sensors vary widely like this, the interface of the gas sensor 60 according to the first embodiment, the interface of the two gas sensors 2060A and 2060B according to the second embodiment, and the interface of the sensor unit 3080 according to the third embodiment are difficult to match. In view of this, in the first to third embodiments, the connection interfaces of all the chambers 52, 2052, and 3052 for the main body 10 are configured to match perfectly. In other words, the chambers 52, 2052, and 3052 are interposed between various gas sensors and the main body 10 to function as adaptors that ensure connection compatibility. This enables the three types of gas detection modules 50, 2050, and 3050 to share the main body 10.

The gas detectors 1 and 2001 according to the first and second embodiments are configured so that the main body-side computer 34, the base-side computer 74 or 2074, and the sensor-side computer 64 or sensor-side computers 2064A and 2064B are connected to each other in series. With the three types of computers thus connected, the main body-side computer 34 can multiplex commands and transmit the multiplexed commands. The base-side computers 74 and 2074 can distribute the multiplexed commands to the gas sensor(s). Similarly, the base-side computers 74 and 2074 can multiplex the measurement results (outputs) of the gas sensor(s) and transmit the multiplexed measurement results. The main body-side computer 34 can separate the multiplexed outputs to individually display the measurement results of the gas sensor(s) on the display device. The multiplexing of commands and signals can reduce the number of pins of the main body-side connector 26A.

The sensor-side computers 64, 2064A, and 2064B can normalize (use a common protocol for) the measurement results (outputs) of the gas sensors. This can simplify the signal processing of the main body-side computer 34 and the base-side computers 74 and 2074.

The gas detectors 1 and 2001 according to the first and second embodiments include the heater power supply unit 210 serving as the additional function supply unit in the main body 10. The supply voltage of the heater power supply unit 210 is controlled by the main body-side computer 34. In other words, the main body-side computer 34 functions as an additional function control unit that controls the additional function supply unit. As a result, the gas detection modules 50 and 2050 and the gas sensors 60, 2060A, and 2060B mounted thereon can be reduced in size since the heater power supply and the voltage control function that tend to be large in size do not need to be built in the gas detection modules 50 and 2050. Consequently, only the gas sensors 60, 2060A, and 2060B can be replaced on the gas detection modules 50 and 2050. While in the first and second embodiments the additional power such as the heater power is described to be supplied as the additional function, the present invention is not limited thereto. For example, depending on the intended purposes of the gas sensors, a high-frequency signal, an optical signal, or the like may be supplied as an additional signal (additional function).

The present invention is not limited to the above-described embodiments, and it goes without saying that various modifications can be made without departing from the spirit and scope of the present invention. The entire disclosure of Japanese Patent Application No. 2023-175124 filed Oct. 10, 2023 including specification, claims, drawings, and summary are incorporated herein by reference in its entirety.

### REFERENCES SIGNS LIST

1 gas detector
10 main body
14IN suction port
14OUT exhaust port
15 lever
18 pump
20 flow sensor
22 main body-side inflow path
24 main body-side outflow path
26 main body-side circuit substrate
26A main body-side connector
27 main body-side auxiliary substrate
27A flexible wiring
34 main body-side computer
49 main body-side engagement portion
50 gas detection module
52 chamber
54 internal space
55 chamber-side engagement portion
58 sensor insertion port
60 gas sensor
61 sensor housing
62 gas detection unit
62H heater unit
63 sensor-side circuit substrate
64 sensor-side computer
65 sensor-side connector
68 sealing surface
70 sensor base
71 base housing
73 base-side circuit substrate
74 base-side computer
75 connector
77 connector
79 base-side engagement portion
102 first determination path
104 second determination path
106 first base power path
108 second base power path
108X heater power path
110 heater power path
112 first communication path
114 reset path
116 third determination path
118 second communication path
130 analog output path
202 first determination power supply unit
204 second determination power supply unit
206 first base power supply unit
208 second base power supply unit
210 heater power supply unit
220 third determination response unit
702 first determination response unit
704 second determination response unit
706 third determination power supply unit
2001 gas detector
2050 gas detection module
2052 chamber
2060A first gas sensor
2060B second gas sensor
2063A first sensor-side circuit substrate
2063B second sensor-side circuit substrate
2064A first sensor-side computer
2064B second sensor-side computer
2065A first sensor-side connector
2065B second sensor-side connector
2070 sensor base
2071 base housing
2071A first sensor insertion opening
2071B second sensor insertion opening
2073 base-side circuit substrate
2074 base-side computer
2075A connector
2075B connector
2077 connector
2702 first determination response unit
2704 second determination response unit
2706 third determination power supply unit
2707A first sensor-side first determination response unit
2707B second sensor-side first determination response unit
2708A first sensor-side second determination response unit
2708B second sensor-side second determination response unit
3001 gas detector
3050 gas detection module
3052 chamber
3054 internal space
3055 chamber-side engagement portion
3058 unit insertion port
3060 gas sensor
3073 unit-side circuit substrate
3074 unit-side computer
3077 connector
3080 sensor unit
3081 unit housing
3090 heater internal control unit
3130 analog output path
3402 determination input processing unit
3404 sensor determination processing unit
3406 auxiliary determination input processing unit
3408 auxiliary sensor determination processing unit
3410 base power supply processing unit
3412 path function switching processing unit
3414 control protocol switching processing unit
3416 sensor setting information reading unit
3418 output demultiplexer processing unit
3420 input multiplexer processing unit
3422 additional function processing unit
3702 first determination response unit
3704 second determination response unit
3706 third determination power supply unit
6402 normalization processing unit
6404 type output processing unit
6406 requested specification output processing unit
7402 input demultiplexer processing unit
7404 output multiplexer processing unit
7406 type output processing unit
7408 requested specification output processing unit
7410 normalization processing unit

### CITATION LIST

Patent Literature 1: Japanese Patent Application Laid-Open No. 2023-076338

What is claimed is:

1. A gas detector for detecting gas, the gas detector comprising:
a main body including a main body-side inflow channel through which the gas flows in and a main body-side outflow channel through which the gas flows out;
a chamber that is separably disposed on the main body and includes a chamber-side inflow channel detachably connected to the main body-side inflow channel, a chamber-side outflow channel detachably connected to the main body-side outflow channel, and an internal space interposed between the chamber-side inflow channel and the chamber-side outflow channel;
a gas sensor that is separably disposed on the chamber and includes a gas detection unit to be in contact with the gas passing through the internal space; and
a sensor base that is separably disposed on the gas sensor and the main body, and that includes a sensor receptacle connector detachably connected to a sensor-side connector of the gas sensor and a main body receptacle connector detachably connected to a main body-side connector of the main body to thereby electrically connect the gas sensor and the main body.

2. The gas detector according to claim 1, wherein:
the chamber includes a sensor insertion port into which the gas sensor is inserted;
the gas sensor includes an annular sensor-side sealing surface capable of abutting against the sensor insertion port; and
the internal space is enclosed by the sensor insertion port and the sensor-side sealing surface abutting against each other.

3. The gas detector according to claim 1, wherein:
the chamber includes a chamber-side engagement portion;
the sensor base includes a base-side engagement portion to be engaged with the chamber-side engagement portion;
the chamber is fixed to the sensor base by engaging the chamber-side engagement portion and the base-side engagement portion with each other; and
the chamber is made movable relative to the sensor base by disengaging the chamber-side engagement portion and the base-side engagement portion.

4. The gas detector according to claim 1, wherein:
the gas detector includes the gas sensor in multiple units;
the gas detection units of the gas sensors are in contact with the gas passing through the internal space of the chamber; and
the sensor base includes a plurality of sensor receptacle connectors corresponding to the gas sensors.

5. The gas detector according to claim 4, wherein:
the sensor base includes a base-side computer; and
the base-side computer includes an output multiplexer processing unit that multiplexes outputs of the gas sensors and transmits the multiplexed outputs to the main body via the main body receptacle connector.

6. The gas detector according to claim 5, wherein:
the gas sensors each include a sensor-side computer; and
the sensor-side computer includes a normalization processing unit that normalizes the output of the gas detection unit.

7. The gas detector according to claim 4, wherein:
the sensor base includes a base-side computer; and
the base-side computer includes an input demultiplexer processing unit that separates multiplexed input signals transmitted from the main body via the main body receptacle connector and distributes the separated input signals to the gas sensors.
